SEQUENCE LISTING (76 sequences)

SEQ ID NO: 1 - DNA, 1000 bp, Komagataella pastoris
tctccaccgtcttgatactttctgaggtgcacagaccagaggattgtgcgaaccctaaatagttgtatgaactcaaattcaagcgctctgtgacggtgccggaataattataggtattgttgtaatcgtcactggttctgtcgtaacatttgatgtacctaccgggaactccacatatgggccttgcaaaacagtcatctaatcttgttttcaaacgacgcgtgtagaaactttcaaaattggcataccatggagcaataccatctttcaccatcacatctctgaatagttcagggtgaaacaacttcccaaagaagtctctaatatggcctattatgatcaaaataaggtaattaaggtatgttgcaatcaggatgaaatatggaggttcgtcctcgatgggaactggaagcggctcgccagggttatgcttagagacaaagagccattctttactggtcaaggacccaaattctttctcggccttctcttgtggtgaatcattgtctatgagagcatctgggatagtttttgacatgatctttttcagcacggctatatagcaaaaagcaaaaaaaaagaccgaatggaattatatggtctaaaaaaacaaactggtggtaaaataaaaaaaaacgactggtgggcggtttcaaaggagactaatgatcttctatgcccgcggaaataaatagtactccaacgactgaactcagcggtattaaagtttgtgaataaaattacaaggcttagaaagcttggttggtctttcggtatctgtagatggtagagttttgagaacatttcatttccacagtaaccaacgaacacgacccgtgacttccgggggttggcagatgttaacgcgcgcgtggtagaagtttatcttgggaggtgctagagggtgctcttggccttgttcgctggggggaagtgtttgtagttaacgtacaactcctcatgactggggatcagaatttcaacttgatttgccgctaat

SEQ ID NO: 2 - DNA, 1007 bp, Komagataella pastoris
cggttaataaaaataatgatttacatttaagaagtaacagcacatatatactgtaagattaactttgcgtaccctaaattttactaataaacttaacgggttgccatagccttggtaaccacacgtttcaatgccaattcagcttttctgaagtcatcaccggaagcatcttctagctccaaagcagccaaagcgtcagacaaagcagactcgatcttgtccttagcacttctctttagcttggaagacaaaattgggtcagtgatggtagactcaatggaggaaacgtaagcctccaacttctgtttggattcgtgacggttagcgaagtcctcgtcagccttcttgaacttgtcagcatcgttgatcatcttttcgatctcggaagaagacaatctaccaatagagttagaaatagtgatgttggcagatcttccggtagacttctcgacagcggtaaccttcaagataccgttggcatcaatctcaaagatagcctccaacactggctcaccagcagacataggaggaatgttcttcaagtcgaactcacccaacaaggtgttctcagaacagttgacacgctcaccctggtaaactgggaattgaacagtggtttggtggtcgtcaacagtggtgaaagttcttctcttgatagttgggacagtggtgtttcttggaacaactggggcaaagacgttaccttgcatggcaacacccaaagaaagagggataacatccaacaacaacaagtccttggtctcttcagaggtagattgaccggtcaaaatagcaccttgaacggcggcaccgtaagcgacagcctcatcagggttgatggatttctccaattgcttaccatcgaagaagtcagacaacagcttttggacctttggaattctggtggaaccaccaaccaagacgacgtcatcgaccttggatttctcgatctttgagtccttcaaaacttgttcaacaggctccaaagtagacttgaacaaatcagc

SEQ ID NO: 3 - DNA, 915 bp, Komagataella pastoris
atgtccattgtgaacactgatctggacggaatcctacctttaatcgccaaaggaaaggttagagacatttatgcagtcgatgagaacaacttgctgttcgtcgcaactgaccgtatctccgcttacgatgtgattatgacaaacggtattcctgataagggaaagattttgactcagctctcagttttctggtttgattttttggcaccctacataaagaatcatttggttgcttctaatgacaaggaagtctttgctttactaccatcaaaactgtctgaagaaaaatacaaatctcaattagagggacgatccttgatagtaaaaaagcacagactgatacctttggaagccattgtcagaggttacatcactggaagtgcatggaaagagtacaagaactcaaaaactgtccatggagtcaaggttgaaaacgagaaccttcaagagagcgacgcctttccaactccgattttcacaccttcaacgaaagctgaacagggtgaacacgatgaaaacatctctattgaacaagctgctgagattgtaggtaaagacatttgtgagaaggtcgctgtcaaggcggtcgagttgtattctgctgcaaaaaacctcgcccttttgaaggggatcattattgctgatacgaaattcgaatttggactggacgaaaacaatgaattggtactagtagatgaagttttaactccagattcttctagattttggaatcaaaagacttaccaagtgggtaaatcgcaagagagttacgataagcagtttctcagagattggttgacggccaacggattgaatggcaaagagggcgtagccatggatgcagaaattgctatcaagagtaaagaaaagtatattgaagcttatgaagcaattactggcaagaaatgggcttga

SEQ ID NO: 4 - DNA, 487 bp, Komagataella pastoris
ttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 5 - DNA, 500 bp, Komagataella pastoris
tcatcgggggtataatacatgtattttgatagaactttgatattattaattatctgattgaattgtaggtccctatgcttttcagaaattgtgccaccatttttcattagttgagcactataaaacagttttcaagattgtagtcgtctaactcctcgcttggttgggcggtaggagttcagtcggaagataaagatctcttcgcgataaggtttagactacattcagtttctccttgatctattacatactcccaagtatgactaacgatgctccgttatgggttctaggctatggttctctaatatttaagccgcctcctcatgcgacacataagattccaggcaaaatctcaggttttgtcagacggttttggcagagttctacagatcatagaggtacaccggagagcccaggaagagtggtaaccttaattccgtacgcagatatagtaagggatcaccggtttctagaaagtgttcattactttgagtgtaacg

SEQ ID NO: 6 - DNA, 6655 bp, Komagataella pastoris
ccaatcaaacaaggtgacttgcgcgaagcaatgatttgtggatgggctgcggtatggcagcataacaatgcaacgctatttcagaaattgtaaagtgtaaaggaaatattcaaccctataaagaatctaccgaaacgtaggataaataatttccacagagtacacttttggtttttatggacggcgttgagttgcacagatgatggaatattgtgataaaatacgctatataattttggagaccctaagatcaagtggactaggtcgattgcgtatattagggtattaaagacttacttaattctataagagctcggcaagctatattctcagttccttcgcagtcgaccttcagtgcccaaaactttaaagtggacgggtgttttctaaagattgctattgttcaattgcccggttttttaaccgctgaaaaccagtggcaagagtggactacgtgcgcattgcaatttgtataatccatataattttgttccacggccttaagttattgtactgtttgctcaattttgaagcggaatatccgtcctagtctcgaatccttatttaagtaccggtatcatttgcatatcctctcagccctcaaacgcccattctttatagtaatggttatgtattaatcgactacgatgattgactcagggaggcacgaatagtataatttgtaacttgcagtgggaagctaatttcgggtgtcttgaaagacgaggttaaaaatttgaacccaaaccgattttaccggagatcctacctcaaattagaacaactttaaagctttcaaggaattgaaattccatctttatagaccggtaattctttgaagagtaatcaatgtaagagcaagttctaaagagaggatccaaattccttctgatagcaattgtaaggagctgcaaagttcaacataatatttgcgatagtaaccgtcctacggatacttaaatcggtatcaaagagtgaccaatcctgaatcagtgtagtgcaaaaagaaataacgatgataaacgaatcaagattctgatgagttgtccaagacggggctatcataatatttaaaaaatagaacctactaatttaacaaggtaaatctcatagatttaagtttcacgactagcttagctaccactcaaataagctcccgcttgttcattcggctggggtctccaatctccatctccaatggatattttttttttaagtggagagagatgttcagtaaactgtgagttcagttgtcagaagcaaccaatatttaccatggagacgaataagcagatattatatactctatttttacagcccttttgtataatcctcagggatggttggcgcttccctgtgttccaagtaaccacactcttcaagttggaaatgcaaaaggtcccaatgcatgctcccaagtcatactgaatacagagatagattctttgaattggatcgatctctaatgaaattcatggatgagaagagagtgttaaaaactttgccccaactaaatactttatctgaatgaatcattaaagaacacgacaagttcagtatgttgcttggagcctccgattcacagtcttgtaagtagtgcgtgttttcaaatatttgcattagcaatccctccaagtagcccacatagggaccaaaaaaatagtatagtttgttttggggggaagggagtaggaattgctttcgaaatggttaactcctgcttagtttcatttaaaaaatgaatagagaagtagatacagaatactttgtaagactgcttacatgaatgatgccagagagtgtgccgaaagatctacatgctactaaatttactatcaatagggataaaatgtattaggctacctactatgccgtattggaaaggcgaagtgctttgtatgcaagggattggggcataggcaaacggatctatgacaagagatacgaaattacttctgttacccactctgctcctaccaaaccaaatatatctgtgaccaaaaatatcatttgtcttgtgctccacatagcggccaggctcaaagagaacactagatctgctttcagacattaccgcgtgcaaatatgacaagtcactaggtgatgaactagtcgataaaaccaaaaacgatctgatcaataacttgacaacatcgttcttatcggatttctagctcttgaatagtcaaatgctgagttcaattttaaagtgaatatttttcaccgaagtcgattttagcccattagtcaaacaacagaagagaaaagacaaaccttctttttcatgacacatgaaacaaatcacaaaaattgagtcatattacggtttcaatttctgctgtttgtataagtcattggctggcaaacacatctcctggcctcatactgaaaatatatgaagataactgcttctaccaatttcaattgatcaaggttctacccatacgtatagttccagagcatgatgttagggaaacttacagggatgatgtgggccacagcataatatcaacaatacctaacaatcggtatttaattcatagatattcaataccaattctccacgatgagaaggggcgactcctcttcttggacgttctgaaaatacaggtacgctcacgtcagtgcctgagcacatacagcgacgttagtatcaatcttcctgggacatctctaatgctctaaaatgaagaaaaagtaaaattagtatcagttggccatgcgaccaagcctgttggaacgcaaaagcatatccgaattatggttggaaattaccttcgaccttcaaacactaaactcttcacagggctattattccagctattgcagaacttcatacgaatttgggcttgctaaaaattggcaggatcgttcttcaataactgaatacagtattgtacctattcaagcagttagtccatacttagggccttcctagcctgtccagtctaccattaacgaaagttctgagctgtgtctatccaaattaatttgtgtcagaacctacgtaaccactataagtctcataacgtttttgaaatagataaaattagtttgtttgatcattcaagcacattatagataatattccaaaaagaaagtttcaaggtgccccttggacatataaaactttgccacggtgctaaacttaaaagaatgatcttccaaaaatacggaatagaataatctatttttagaagagaagactaaaaatcgagcgatactatgaatgtctgttacaaactgaatttttgcacgtaacgaatactttgaaacaatcctcaaaatcttgttttggtgtttgtcgagtagtctttaccaggccgaatcctgtagttaaaaataattttctatttggacaactaagggtatcccctcaagtttggcttttgtgtaccttgttcggaaaggctccgttctgcaattctgcagcaaccttgtcattagctgtgtaatgctcgctggtgagtagcataagtcattccaatggtaactaccaagcttgtaatataaccaccagcacctccattcagtagtagcaatgagcaaggtcggagattgcttttaagcgaggtttcaaagtaatcttgtagttttgctttgtcgaaaaatggccacggcttacacatacagttcgtccaccagatctttaggcttcatcaacaaagttttcgacttcgttgtctataggttttattctttatcagcgtcagactatgatcgtactagtttgagctgaagcccatcaggatgtttaccactccagttttgtaatttccgcggctatctagggctacgttgtccattgccgagtttatatatgacatcacattaggcagtaattctccatgcttctttttcgataatgttttggtctaggttcgtgcatgaaattttttcttcatccagactatctctgcgctgcactgttcacatagccaactgatactgatttcactttttcttcattttagagcattagagatgtcccaggaagattgatactaacgtcgctgtatgtgctcaggcactgacgtgagcgtacctgtattttcagaacgtccaagaagaggagtcgccccttctcatcgtggagaattggtattgaatatctatgaattaaataccgattgttaggtattgttgatattatgctgtggcccacatcatccctgtaagtttccctaacatcatgctctggaactatacgtatgggtagaaccttgatcaattgaaattggtagaagcagttatcttcatatattttcagtatgaggccaggagatgtgtttgccagccaatgacttatacaaacagcagaaattgaaaccgtaatatgactcaatttttgtgatttgtttcatgtgtcatgaaaaagaaggtttgtcttttctcttctgttgtttgactaatgggctaaaatcgacttcggtgaaaaatattcactttaaaattgaactcagcatttgactattcaagagctagaaatccgataagaacgatgttgtcaagttattgatcagatcgtttttggttttatcgactagttcatcacctagtgacttgtcatatttgcacgcggtaatgtctgaaagcagatctagtgttctctttgagcctggccgctatgtggagcacaagacaaatgatatttttggtcacagatatatttggtttggtaggagcagagtgggtaacagaagtaatttcgtatctcttgtcatagatccgtttgcctatgccccaatcccttgcatacaaagcacttcgcctttccaatacggcatagtaggtagcctaatacattttatccctattgatagtaaatttagtagcatgtagatctttcggcacactctctggcatcattcatgtaagcagtcttacaaagtattctgtatctacttctctattcattttttaaatgaaactaagcaggagttaaccatttcgaaagcaattcctactcccttccccccaaaacaaactatactatttttttggtccctatgtgggctacttggagggattgctaatgcaaatatttgaaaacacgcactacttacaagactgtgaatcggaggctccaagcaacatactgaacttgtcgtgttctttaatgattcattcagataaagtatttagttggggcaaagtttttaacactctcttctcatccatgaatttcattagagatcgatccaattcaaagaatctatctctgtattcagtatgacttgggagcatgcattgggaccttttgcatttccaacttgaagagtgtggttacttggaacacagggaagcgccaaccatccctgaggattatacaaaagggctgtaaaaatagagtatataatatctgcttattcgtctccatggtaaatattggttgcttctgacaactgaactcacagtttactgaacatctctctccacttaaaaaaaaaatatccattggagatggagattggagaccccagccgaatgaacaagcgggagcttatttgagtggtagctaagctagtcgtgaaacttaaatctatgagatttaccttgttaaattagtaggttctattttttaaatattatgatagccccgtcttggacaactcatcagaatcttgattcgtttatcatcgttatttctttttgcactacactgattcaggattggtcactctttgataccgatttaagtatccgtaggacggttactatcgcaaatattatgttgaactttgcagctccttacaattgctatcagaaggaatttggatcctctctttagaacttgctcttacattgattactcttcaaagaattaccggtctataaagatggaatttcaattccttgaaagctttaaagttgttctaatttgaggtaggatctccggtaaaatcggtttgggttcaaatttttaacctcgtctttcaagacacccgaaattagcttcccactgcaagttacaaattatactattcgtgcctccctgagtcaatcatcgtagtcgattaatacataaccattactataaagaatgggcgtttgagggctgagaggatatgcaaatgataccggtacttaaataaggattcgagactaggacggatattccgcttcaaaattgagcaaacagtacaataacttaaggccgtggaacaaaattatatggattatacaaattgcaatgcgcacgtagtccactcttgccactggttttcagcggttaaaaaaccgggcaattgaacaatagcaatctttagaaaacacccgtccactttaaagttttgggcactgaaggtcgactgcgaaggaactgagaatatagcttgccgagctcttatagaattaagtaagtctttaacaccctaatatacgcaatcgacctagtccacttgatcttagggtctccaaaattatatagcgtattttatcacaatattccatcatctgtgcaactcaacgccgtccataaaaaccaaaagtgtactctgtggaaattatttatcctacgtttcggtagattctttatagggttgaatatttcctttacactttacaatttctgaaatagcgttgcattgttatgctgccataccgcagcccatccacaaatcattgcttcgcgcaagtcaccttgtttgattgg

SEQ ID NO: 7 - DNA, 28 bp, Artificial Sequence
  Primer 1
actgctgcagtctccaccgtcttgatac

SEQ ID NO: 8 - DNA, 30 bp, Artificial Sequence
  Primer 2
tcgcggatccattagcggcaaatcaagttg

SEQ ID NO: 9 - DNA, 34 bp, Artificial Sequence
  Primer 3
cgccggatcccggttaataaaaataatgatttac

SEQ ID NO: 10 - DNA, 33 bp, Artificial Sequence
  Primer 4
tgacgtcggtaccgctgatttgttcaagtctac

SEQ ID NO: 11 - DNA, 28 bp, Artificial Sequence
  Primer 5
atctggtaccgggtgctatcgttttgtg

SEQ ID NO: 12 - DNA, 29 bp, Artificial Sequence
  Primer 6
acatggtaccccatactgagccaaaagac

SEQ ID NO: 13 - DNA, 32 bp, Artificial Sequence
  Primer 7
ttaggcgcgccttttttgtagaaatgtcttgg

SEQ ID NO: 14 - DNA, 47 bp, Artificial Sequence
  Primer 8
ttaactagttgtgttttgatagttgttcaattgattgaaatagggac

SEQ ID NO: 15 - DNA, 30 bp, Artificial Sequence
  Primer 9
ttactcgagtcatcgggggtataatacatg

SEQ ID NO: 16 - DNA, 35 bp, Artificial Sequence
  Primer 10
ttattaattaacgttacactcaaagtaatgaacac

SEQ ID NO: 17 - DNA, 40 bp, Artificial Sequence
  Primer 11
ttagcggccgcccaatcaaacaaggtgacttgcgcgaagc

SEQ ID NO: 18 - DNA, 30 bp, Artificial Sequence
  Primer 12
gaaatcagtatcagttggctatgtgaacag

SEQ ID NO: 19 - DNA, 28 bp, Artificial Sequence
  Primer 13
aaaattagtatcagttggccatgcgacc

SEQ ID NO: 20 - DNA, 38 bp, Artificial Sequence
  Primer 14
ttatctagaccaatcaaacaaggtgacttgcgcgaagc

SEQ ID NO: 21 - DNA, 855 bp, Artificial Sequence
  Zeo
cccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 22 - DNA, 795 bp, Artificial Sequence
  G418
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 23 - DNA, 720 bp, Artificial Sequence
  GFP
atggtttctaagggtgaagagttgttcactggtgttgttccaatcttggttgagttggacggtgacgttaacggacacaagttctctgtttctggtgaaggtgagggtgacgctacttacggaaagttgactttgaagttcatctgtactactggtaagttgccagttccatggccaactttggttactactttgacttacggtgttcagtgtttctccagatacccagaccacatgaagcagcacgatttcttcaagtctgctatgccagagggttacgttcaagagagaactatcttcttcaaggacgacggtaactacaagactagagctgaggttaagttcgagggtgacacattggttaacagaatcgagttgaagggtatcgacttcaaagaggacggaaacatcttgggtcacaagttggagtacaactacaactcccacaacgtttacatcatggctgacaagcagaagaacggtatcaaggttaacttcaagatcagacacaacatcgaggacggttccgttcaattggctgaccactaccaacagaacactccaattggtgacggtccagttttgttgccagacaaccactacttgtccactcaatccgctttgtccaaggacccaaacgagaagagagatcacatggttttgttggagttcgttactgctgctggtatcactttgggtatggacgagttgtacaagtaa

SEQ ID NO: 24 - protein, 932 aa, Komagataella pastoris
MSEELKEPKNRAPLPNFSHLVYDLFEKLDTCVKSPHIPESLRSRKTKIIEQFVQKWRFEIGDNIYPASRLIFPNLDKRHYNIKDFTLIKFILDIFQVPKDSEDTKILRNWKGKYENRMRSNKNDLAQLLTGVIKARRGAVLDRPEISIDRLNELLDTMTRPETKPSDQKKILTEIFNHVDNIELKWFLRILLKRKLMGGMDFTFFRQWHPDAVELLQLVNDLKTVFWELTDRLIPLPEAGRVIRVGRAFRPQLAMRPNRTYDQIATLFDNNFYIEDKLDGERILMHISLNDTTQQHEFKYFSRSGTDYTYLYGSNYEEGAFSPFIKGCFIDLKDDTKEIRSMVLDGEFLVYDNKRNVILPFGALKSASLTRLMEEKNDSEDSQETQVYFVVYDLVFLNGTSFQDKALKVRRNLLNKILANPIPNRIEVIKYTEGHVGSDIEAAMRSAIHDNREGIVVKNPKSKYYIASRNLNWVKIKPEYLEEFGENIDLIILGKRKGLKNAYICGLRESLGNDRYRFLSLARIANGFTGQMYEEIEHSLGSKWVNIRKTQPPYPYIDLGEISSMVDYWVDPKESVVLEIKARSIDIGDGGKRYAAGTTLYNAYCVRIRSDKDFLSCSTFADYKEIKTSKSRHKTEKTHSLMSKRKRTIVTEPILAKQSVSSKSSIFKDYIFYVLSDMLGEKRYSIAQLEALLVENGGRITKNDCVALADGERLVLLSDKITFKVTEYFNKGLNVFRPGFILKCLQLKTIIPLQINDILLADESLIEKVKKNSDRFGDSYTSPIDRKELRMLMKGIPKIKLEHPLKFPLLYNLFKGLKFYIARPCYRSPFLTNVMSPYLLEVIVILGGFLTTDIFQCSFIIVDTSTTFNYSWLTEEATIQDEVGSLRKKLGDDLPIVKGVLDKVPRIVEKEWVFKCIQEGTIVAADGYNYIG

SEQ ID NO: 25 - DNA, 2799 bp, Komagataella pastoris
atgagtgaagagttgaaggagccaaaaaacagagcccctttacctaatttcagccatctagtttatgaccttttcgaaaaattggatacatgtgtcaaatcgcctcatattcctgaatctctcagaagtcgaaagaccaagataatagagcagtttgtccaaaaatggagatttgagataggtgataacatatatcctgcctcaagacttatttttcccaatttagataagcgtcactataatatcaaggattttacattgatcaagttcatattggacatcttccaagttccaaaagactccgaagatacgaagattttacgtaactggaaagggaagtatgagaatagaatgcgctccaacaaaaatgatctagctcaattactcactggagttattaaagctaggagaggtgctgttttggatcggccagaaatttctattgacagactgaacgaacttttggatacaatgactcgccctgaaacaaaaccgtctgaccaaaaaaagattttgacggagattttcaatcatgtagataatattgaactgaaatggtttcttcggatcctactgaaaaggaagttaatgggtggtatggattttactttttttaggcagtggcatccagatgctgtagaattacttcaactggttaatgacctgaaaactgtattctgggaactgactgatcggttgattccattgcctgaagcgggcagagtcatcagagttgggcgtgcattcaggcctcaactggcaatgcgacccaatagaacttacgatcaaatagctactctatttgacaataatttctacattgaagataaactagatggcgaacggattttgatgcacatcagcttgaatgacacaacccaacaacatgaattcaagtacttttcaaggagtggaactgactatacatatttatacgggtcaaactacgaagaaggtgcattctctcctttcatcaaaggttgcttcatagatctcaaagatgacacaaaagaaatccgatctatggttttggatggggagtttctcgtttatgataacaaaagaaatgttattctcccctttggggctctcaagagtgccagtttaacgagattgatggaggagaaaaacgattcggaagactctcaagagacgcaagtgtatttcgttgtttatgatcttgtattcttaaatggtacatcatttcaagacaaagctctcaaggtaaggcggaatttactgaataaaattcttgcaaatccaatccctaacagaatagaagttatcaaatacacggagggtcatgtcggatcggatatcgaagctgccatgagaagtgctatacacgacaatcgtgaaggaatcgtagtgaagaatccaaaatcaaagtattatattgcatccagaaatctcaactgggtcaagataaaacctgagtacttggaagaatttggagaaaatatagatttgattattctaggcaaaagaaaaggactgaaaaacgcctatatctgtggactcagagagagtcttggtaatgaccggtacagattcttgtcactggccagaattgccaatggatttacagggcaaatgtatgaagaaattgaacattcactggggagtaaatgggtcaatataaggaagacgcaaccaccttatccatacattgatctcggagagatatcatcgatggtggactattgggttgatcccaaagaatctgtcgttttagagatcaaggctcgttcaattgatattggtgatggcgggaaacgttacgcagctggtaccactctttacaatgcgtattgtgtaagaattagatctgataaagacttcttatcatgttcgacttttgcagattacaaagaaataaaaacttcaaagagtagacacaaaacagagaaaacacacagtctaatgagtaaacgaaagaggacaatagtcactgaaccaattctcgctaaacaaagtgtatcttcgaagagcagcatattcaaggattacatcttttatgttttaagtgacatgttgggtgaaaaacgatactctattgctcagctggaggctttgctggtcgagaatggtggcagaataactaagaatgattgcgtagctcttgcagatggcgagaggcttgttttacttagtgacaagattactttcaaagtcacagagtactttaataaaggtctaaatgtctttcgaccaggcttcattctgaagtgtcttcagctgaaaactataattcctctacaaattaacgacatattactggcggatgagtctttgatagaaaaggtaaagaaaaactccgaccgatttggggatagttatacctctcccattgaccgtaaggagttgcgaatgcttatgaaaggtatacctaagataaagttagaacacccactaaaatttccactactgtacaacttgttcaagggcctgaagttttatatcgcaagaccatgctatagatctccatttctgacaaatgttatgagtccatacttgttagaagtaatagtgattctaggaggatttcttacaactgatattttccaatgttcatttataatcgtggatacttccaccacattcaactattcctggttgactgaggaggcgaccatacaggatgaagttgggtctctaagaaaaaagctgggtgatgatctaccaatcgtcaaaggtgttctagataaagtaccccggatagtagagaaagagtgggtgttcaaatgtatccaggaagggacaatcgttgctgcggatggctataactatatcggttaa

SEQ ID NO: 26 - DNA, 94 bp, Artificial Sequence
  Primer 15
ttaaagctttctagagcggccgcggcgcgcctgcggcctgtttggccttaattaagcgatcgcggcctgtttggcccccacacaccatagcttc

SEQ ID NO: 27 - DNA, 31 bp, Artificial Sequence
  Primer 16
ttagaattctcagtcctgctcctcggccacg

SEQ ID NO: 28 - DNA, 30 bp, Artificial Sequence
  Primer 17
ttaactagtatggtttctaagggtgaagag

SEQ ID NO: 29 - DNA, 38 bp, Artificial Sequence
  Primer 18
ttactcgagttacttgtacaactcgtccatacccaaag

SEQ ID NO: 30 - DNA, 22 bp, Artificial Sequence
  Primer 19
atgattgaacaagatggattgc

SEQ ID NO: 31 - DNA, 22 bp, Artificial Sequence
  Primer 20
tcagaagaactcgtcaagaagg

SEQ ID NO: 32 - DNA, 52 bp, Artificial Sequence
  Primer 21
ttcaatcaattgaacaactatcaaaacacaatgattgaacaagatggattgc

SEQ ID NO: 33 - DNA, 52 bp, Artificial Sequence
  Primer 22
atcaaaatacatgtattatacccccgatgatcagaagaactcgtcaagaagg

SEQ ID NO: 34 - DNA, 82 bp, Artificial Sequence
  Primer 23
agactttaaatttaatttatttgtccctatttcaatcaattgaacaactatcaaaacacaatgattgaacaagatggattgc

SEQ ID NO: 35 - DNA, 82 bp, Artificial Sequence
  Primer 24
caatcagataattaataatatcaaagttctatcaaaatacatgtattatacccccgatgatcagaagaactcgtcaagaagg

SEQ ID NO: 36 - DNA, 24 bp, Artificial Sequence
  Primer 25
aggagttagacgactacaatcttg

SEQ ID NO: 37 - DNA, 164 bp, Artificial Sequence
  PARS1
tcgagataagctgggggaacattcgcgaaaatgaaacaagtcggctgttatagtatatttattataatattgaaagatctcaaaagactacttatttttgaatgaaccaagtatgaaatcaacctatttggggttgaccaaaataagtaaatattaattgtcga

SEQ ID NO: 38 - DNA, 37 bp, Artificial Sequence
  Primer 26
ttagcggccgcttcgagataagctgggggaacattcg

SEQ ID NO: 39 - DNA, 40 bp, Artificial Sequence
  Primer 27
ttagcggccgctcgacaattaatatttacttattttggtc

SEQ ID NO: 40 - DNA, 30 bp, Artificial Sequence
  MCS
ggtacccctagggatatcacgcgttgtaca

SEQ ID NO: 41 - DNA, 1000 bp, Komagataella pastoris
catgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 42 - DNA, 32 bp, Artificial Sequence
  Primer 28
ttaggcgcgcccatgttggtattgtgaaatag

SEQ ID NO: 43 - DNA, 35 bp, Artificial Sequence; identical to SEQ ID NO: 16
  Primer 29

SEQ ID NO: 44 - DNA, 100 bp, Artificial Sequence
  Primer 30
acgacttttaacgacaacttgagaagatcaaaaaacaactaattattcgaaacgggtacccctagggatatcacgcgagtgttatgtcaagttctggacc

SEQ ID NO: 45 - DNA, 24 bp, Artificial Sequence
  Primer 31
gaaaacgtactcagtggttttctc

SEQ ID NO: 46 - DNA, 1106 bp, Ogataea angusta
acgacttttaacgacaacttgagaagatcaaaaaacaactaattattcgaaacgggtacccctagggatatcacgcgagtgttatgtcaagttctggacctgcgattgaggtgtccacagaattgaagcagaacgtttttggcgaagacccagtgatacccgaatttgccaaagatacccaatctggcagttctttcgcaaacattactcagtggaaagagctcggtgcatatttagagggcttacaaagaatcacacctggttcctctgactcagaaaacagcgatctacaatccaaaaatccgctctccagaccggttgtgtatcgtatttgcaaccattgcgacagacctatcttggagaaatatttagcagatcatctcaagagctgcgagcaggaaaaacgtaacaaaaccttaacggcagaaaaatccaataccgccattacgaataacgccatgaagaaacggcggttggacgaagctgcagaaaatctgtcaagctcacttaactctccggatgttgaggaaaacaaaatgaacggtgccaacggggcgtccaaaaagcagaaagtcgcgaaagtgcccaaggaaaagaaagtcaggaaaagcgtcaagccaaaaattaccggaaagccaaagggacctgttgatgtggagaaacagtgtggtgtcccgctgccgaacggtggtttctgtgcgcggtccttgacctgtaagacgcattccatgggagccaaacgagccgttccggggagaagcgcgccgtacgaccagctgcttgctgcgtatcagcgcaagaaccaggctaaaatcggtgctgctgctgctgctgctcagcaagctcaagacgaccttatgcatggttcgtctgttccgctggacgacgaagaggagacacatcaggttttggatggcgtgacgagatcgacgccatggcctttggagcggaaagtgataatgcctactaaaatacgcaactcatttattcgcatgagagaaatgtttgctggtgctatactgcctagaatgccgtccaatccgctgggacaaatgcaagggcgtactgcggtcgtggacacagagaaaaccactgagtacgttttc

SEQ ID NO: 47 - DNA, 23 bp, Artificial Sequence
  Primer 32
tattcgcatgagagaaatgtttg

SEQ ID NO: 48 - DNA, 25 bp, Artificial Sequence
  Primer 33
agatctctcacagatactgtttgtg

SEQ ID NO: 49 - DNA, 2503 bp, Ogataea angusta
tattcgcatgagagaaatgtttgctggtgctatactgcctagaatgccgtccaatccgctgggacaaatgcaagggcgtactgcggtcgtggacacagagaaaaccactgagtacgttttccctgtgcggtctcagcatcaacggatggcaccctcgacgcaggtgcggtcggccgcacctatggccaagccaagtccccagggtgccgttgatgttcattcgcaaatctcggcccaggcccagctgctcgctcagcagcagcagcagctcagacttgcccaggccaagaaacagcagcaacaagcacagatgcaagctaaataccaggcccagcagctggctgcaggggtcgttcctcagcaggataaacacatgcaattgactccgcagcaacagcagcagatgatgatgaggcggcggatgtatttgcaacagcagtcgcaacaactgcggcaacaacagcagcagtatattaacccccagtacaagagtcaacaggagatgatgatgaatcaacgccagcaatagtgctgcaacaggctggatctacgatcttggggttttcgggtctgtcgaatcgtcttccagttgcgaagctctcggcttgggttgtaagggctcaatacgatctgaaaatctgattctgaaaaaggaggggtgtttccagttcccgttttcatcgaagttctccggactaggcggtgtgtatttgatgagctgatattctttaagaacatagaagctcatgagaaacccgcctagaactccgatgaccacaacgttaaatcgtccgttaactgttttttgtaccatttggtaatttgtttttctaataaaactccacttttaaaaagaagtaatctttattttcttatcatgtcacttccaaagagaattattagagtaagtaaacaaacatgttcacgtgctaataccaggaaactgaacggctagtatcagaccccgtgccgggtatcacagcagtgccacatgaaggcaacttgcgctacttcaacgtgaccatcgacggtccctccggttcgccatacgagggaggcaaattcaagctggagttgtttctgccggacgactacccaatgctggcccctaaagtgcgttttttgaccaaaatataccatccgaacattgacagactgggaagaatctgtctcgacgtcctaaaggccaactggtctccagcattgcaagtgcgcaccattttgctgtccatccaagcacttctgagctcgccaaacccagacgacccactagcgaacgaggtggctgaggaatggaaaaataacgagcaaaatgccatcaaaaccgccagagagtggactgaaagatttgcacagtgattacaacctctaaatacgagcttcgcgaattccaccggtgagcaacacttcaccttcccacacacttgccagcacgtcactgaagtcgtcaacatacacaatttcgagtcccaaagtctgatagacccacttttcaggttctccagcgtgcgtgcggccctccttaagaagctgctcttcctcgtcgaggaatttagcaaaaagctctttggctagctccctgttcctcagattatagatgaagtcttcaatcacatccttgcggttctgccgtggaagtagcaccttgttcaccttgcctgcaaggtgtgcacccagtagtttttctctcactcctccgattgggagaactttaccagtaagcgtgatttctcctgtcatggcgagcgtttccggcacttttttgcgcaaaaccagggacatcaaacacagtgtcatggtgattcccgccgaaggcccatctttcgagatagcaccttcgggaacgtgcaaatgaacgctagtgttctcatatcgtttgagcagttcgtctctaaattcttgagtgccggccaccaaattgttgtgtagcaggtatccaacaagggtatttgctatctgggagctctccaacaacacctctcccaggcgtccagtgcagttcattttctgcgatccaggaagaccgatcatctcaaatcgtaaaagagatcccgacccgtcagagttataggataatccgttgaccacgccgtaccctttctgaaatttggtctcacctgccgctgtagcacgaagatgtgacggactgccaatatattttgcgagatcgtgcacagtcaccgttttggtggcctcgcccatttgcctctcaacagcctttcctcgacagatagccgcaatcagccgttcaagatttcgaattcctggctcgctggtgtagtgcgtggccatcttcaggatcgtctcgtcgtccatggccaccgcgtcactagaaagtcctgcacgttccaattggcgcggaataaggtatttcttggaaatctcaactttctccatataattgtatcctgcaagttcaattacctccatcctgtcacgcaaaggatcacttagctgccatagatcattcgaggtgcacacaaacagtatctgtgagagatct

SEQ ID NO: 50 - DNA, 25 bp, Artificial Sequence
  Primer 34
ctttctccatataattgtatcctgc

SEQ ID NO: 51 - DNA, 23 bp, Artificial Sequence
  Primer 35
ctacacggattctgtcgagatac

SEQ ID NO: 52 - DNA, 2567 bp, Ogataea angusta
ctttctccatataattgtatcctgcaagttcaattacctccatcctgtcacgcaaaggatcacttagctgccatagatcattcgaggtgcacacaaacagtatctgtgagagatctatagggaagccgatgtagtggtcgtggaagttggtgttttgttcagggtccagaatttccagcagagcagcctcagggtctcctttccgagagttggatccgatcttgtccacctcgtccaataaaatcaccggattcattgactgagagcgccgcagggcctggactattagccctggaattgcaccaacataagttcttctgtggcccttcaggtctgcaaaatcatttagtccaccaacactgatcctctggaactttcgccccagagtggtggcaattgaccgcgccaatgacgttttcccaacgccgggagggcctgtgagaagcaagattggcgctttcagggtggaagccgggcgctctttcgttgtaggctcgtctccagtgccgtatacaaattccggctcgtgtctcggctgtttctgattgtgcagattcaacactgccagatactctagtattctttccttggcactctccatgccgtaatgatcggcgtccagttgctctcgcgcacgtgcaaggtccacattgactgattccacaaaagagtcgagccgctgccacggaagatccatgataatctccagatacgctctcaatacctggtaatccgaggaactggactgcatctgtttgagtctctcaaaatccttcgcaataagtctcttaccgtcctcattaatcagatatttgtccaaagagtcgacaaaatcctggatactcttattttcgtcattgtcctcagccgttctgctcccacggccagtgacccgcatgagacccatctcctcaactagccgacgcaaattccgcagatgatttgagataaattgtgagcgcgacacattgctctgagccttgctgctatggtgatcaagactgctccaaatctccgccaaatagctagtatcgagatgttcttcaaacaccttgttcgcgaacgccacgcatttgttaaatacttcgatacgttctgctcctttggacctcagatacgaaatcttcagctcaattggaaatggaaacagcgcaacaagaacgtcgttgtagtgagcaaagtctcctgacttcctcccgtacgcttgtctcagtcgtttcagacctgcagacacgtctgacgccgacagctgagcattcaaaagcgttgcaagaggcgtgagtcgcaaaagaatgtgccgcgagcgttcctccttgtcctctgagtccaaagtgccctcgtccttgtagtaggttaggaactggtcgatgttgtcgaataaacggatcatgtctttcgtgtcgggatgtggcacagacgtttcctcctgaatctcaacggttgccgtatcctcgtcggattgcacaatatagccccgcgtaacgcccttgaaactcaccacggtcgtctcgctcatctcgtaaaagccgttcaccacacagacggtgcatgtgtccgagtctggatagccaggcacacaggcaactaaatgtttagaagaaagaccaaagcgcgaagcaattgccgcaacacgttctgagcgttccttagctgaccatctcgaagccagatcaagagctttttgcttatcgaaaacaacacgataagtgatgccgggaagaaaaatgaggttgcgctccaaccggtggacaggaagttccacttgccacgctgaagacttggaagacatgctagtggaatatatctgactgaaggctacttttggcgagataatatatagattatcggataagctttcctaatggagcaggagtcgcacacgacttatattttttgctttagctatacaacaacagagaaatgaatacttatgccaacatgccaacactctagaacaagcaatgataccacccgaagcactggctcacttgatgaggtccaaaatttgctgcgtcaggttggatatctcatctgccccgttgaaatcttcgccggttagattgttgaggttcatcttgcctctgttgatttcgctttttattttggacttgatctcctggttatcggattctagtaactgcagcatcgtccacaaggcaatgtgctcgaatgtagagttgcctgagttcagaaaatcggagatgaagctagagatgccgtcgtaattttccaaaattatcgtataatcttgtattcttgagcacaagttggccagggcagcggccgagttaccgctcacttcagggttcgacgatttggtaagcgggatcaacacgtccatgataccaagctcaagcagcttagccttgaggtcatctgccagcgcgagaattgcaaaacatgcagatatttcgctttggacgctttcgggagcttgcaacaccaactctttgcatttcttaaccgcgcccgcttccagtagctccatgcgatttctttcactgctggcagcaaggttcctcaaagtggacacggcgtggcattgtatctcgacagaatccgtgtag

SEQ ID NO: 53 - DNA, 22 bp, Artificial Sequence
  Primer 36
caacaccaactctttgcatttc

SEQ ID NO: 54 - DNA, 23 bp, Artificial Sequence
  Primer 37
agtaggccagtttatcgtacagg

SEQ ID NO: 55 - DNA, 2488 bp, Ogataea angusta
caacaccaactctttgcatttcttaaccgcgcccgcttccagtagctccatgcgatttctttcactgctggcagcaaggttcctcaaagtggacacggcgtggcattgtatctcgacagaatccgtgtagtccaccaaattaaccagaggcttcaagaaacctgcgtcgatgatcaatgcctcgttcatagggtggatcgatatattgcggatgcatgcgacggcagcaagaaccaatggttcgtgctgcgacttcagcaacgagacgagattcggcaagccacccgcgcgcacgatctccaattggtacaatgcatcggaggctaggtttctgagagccaacgtggcctggcattggaccctcgggctcgaagagtccattagttgcaccaactggctgacaagctttggctcggtggtggccagtttttttctattggactcgtccacagcaatgttggacaatgctgtcgtacaatagtactggacgtctgggtcagaggaggaaagcagctgaaccagaactggcaccgacccggcctccacaagctcttttctgttttctagagaatgagtcatgttcagcagggcacctgtggcattccgttgaaccctgaggtctggggacttggcgagtttcgtgagtggaataagagcaccagaggtggcaattttggtcttgttcttatcctgggtagccagatttgtgatacagccaacagcattgcactggacctcaatgttggaactcatcatctgcctgataagaggcactagtccgcccatctccacaattaggaccttgttgtcatcgttgactgccaggttacctaacgcggcacaggctgcccgttggacgtcctggtcagagctctgtagcaatattaaaatcggctccaacacgtctctcgagacggcgcggatgtctttctctgttatttcggcaaatgccaaagctgcacttctctggaggtcgatattgtccgagtacacgagcgtgctaagtgctttcagcggaccatcagagaaaaagtccacctcgccacggttctccaagtattgcaagagagcggaaatcgcgtctctctcgttgtcagctagaagttggggatacaagttgtcatgctcacttttgctacaacaacagcccatttttatgtgttatttccaattgaagacgctcttcttatttgttgggtatgttttcgcagaagtgtctatattggcctcaataatttgttcggttataggtttgccataagatgtaatcttacacgtcttatccgtttgttctgtttaaacaaatttttatgagggcaagatggtagtgagagacacaagataagatacaaaaaatgtcttctagattggtccagcgtgctattttcaagtcgactcgcgagcctacgggcttgtttttggagattaccgcatggagctgtttggtgtttagcaattattacccattaattttcgttcagtacatactcgcaaagttctgcctcttcggctttactgacatggcaggttttcgttcctgcttcacaaacggatcggccttttcgtggtacggcctgtttccaggcctactatctttcgcgcgtgtaggagacttttccctggcgtccttttggttcctgagtctttcgcggtactcttccactttcttgtcgatctcgctgtcttcaagcccgtcatcctcaagcttctctctgtattccattacttttatctccaactgtcgtttgcgctcgtgaaggtcgatatctctgtctcgaacagtagtggcaacctgtttgcgctcgtttttctgccgaagttgccgcctcaaatactgcttccccccactctcatctatacgcgcattgccgtgcaagttgctgaggttacgctgcacgtatccgtttgttccagagcccctcgcggtgctcagaccgattccattgtacatttaaattttatttaatttttgttggcggtgcaaatcgttttagcatgtcgcaacagatgctgagtcgctggattaagcgggagaacagttcatctcccgtgccagacacggccaacaacacagaggcggtggaaaacagccagaagaacggcaagtcgcgttccacgccgtcgactgggtccaaagcgtcgtccatagacgcgttcaggtattccgacactgctggatcgctgacccgctccaaatttgcagataagcttaagggatacactgttgggacccggaaaaggctgtttagcgaagaaggcgaggatgccccgaaaaaggcgaaatcaagcaagttgaccccactcgaacagcagatctacgatctcaaaatgcaacatagagacaaactgctggccattcaagttggctacaaatacaagttctatggcgaggatgctcgtgctgcgtcgcaaattttgaacatcatgttcattccaggacggctgtttttcaccaacagcgatgacctgtacgataaactggcctact

SEQ ID NO: 56 - DNA, 25 bp, Artificial Sequence
  Primer 38
tcaagttggctacaaatacaagttc

SEQ ID NO: 57 - DNA, 70 bp, Artificial Sequence
  Primer 39
agatggctacctgattggacgaggacaccaagacatttctacaaaaaaaatgtgatcacaccagatcttg

SEQ ID NO: 58 - DNA, 2624 bp, Ogataea angusta
tcaagttggctacaaatacaagttctatggcgaggatgctcgtgctgcgtcgcaaattttgaacatcatgttcattccaggacggctgtttttcaccaacagcgatgacctgtacgataaactggcctactgctctattcccgacgtgagacttcatgttcacctgaaacggttgctgagtgccggctacaaagttgccgttgttgaccagaacgagacagcagccatcaagtccaccacagcgtcgaaaaacaagctgtttgagcgacgcatcagtaaagtgtacacttcgtccacgtacatcgacaacgaagacgtgatttctgggggccggttcgttgtcgcgttgacagagactaagaacaaggaaacccctgtgatatctctggttgccgttgacgtttactctgcagacatcatatatgacgagtttgaggacaattttgtgcgcaacgagctggaaactaggctttaccatctcgatccaacggaatttctattaatcggagagatttcgcgtgaaacacagaaagcgttggatctgtttaaacggtacactcgtgagtcggcaactagcttgcggtcggaggccagggatgcaaagacatacacgcagatcgccgatgttctgaacgtcaacctatccgacgaggcctttgatctcgtcgcaaagctttcagtcgctgtccaaggttgttttgcagagctcgtcgagcacatgatcgagttcgaattggccaacgtgtttgatctggtggacaaatacacccatttttccagcgtccatcgatgcatgattttggacgcaaacactctgcgtaacctcgaaatatacaaaaacagcaccaatggccaggagtacggctctctgctgtggatgctggaccatacaaatacccagttcggacgcagagagctaagacgatggatcggcaggccattgacggacagagaagaggtggccaagagagcagactccgtggagagcattatgaaaaactaccagtccgtggccatcgagtcgaccgtcaagcttttgcgcaattgtccggacctggaggcggcgctcagtcgtatccactacggccggtccaaacgcaaggacacttatatgtttctcaaaaaaatgaacgagattctccagttctacggagacctcccagacacctatgctacctctgtgcagaccaacccgtctctacgggagatattcgatgatctcaaaacttcagcctcctctgggctcaaagacttcaggaatttgctggacatggtccactcgccagcagccatcgacgacacgagccccgagcacgtgaccggctatttcaacaccagcttctttgattaccacctgatccaacagcatctggagaacatttctcaggtggagcagcagctggaagccgaactcaaggaaatccgtaaaatcgttggccgtccaggcatgggctatgtgaccaacaacaaggagccgtatttggtggaggtgcgcaacactcaggttgccagcctgccgaaagactggcttaaaatcaacggcaccaagtctgtgtcgcggttcagaactccttcgggagccgcactctacagacagatccagtaccactcagagatgctccagaaagagtgcaacgactgcttcaccaagtttgtcaaaagaatcgacgaatactatctcgacttgaacaaaactattcgacatctcgctgttctagactcactaatttccctgagtgccgcctcctcgctcaacgaaggctatacaaaacctgtgtttgtggactctccgtgcattgatgtgaaaaactctcgcaacccaatttcggaaaacctcaagacatcaactcgttacattcccaacgatttcaagatgtcgcactcagaaggcagaatagccttgatcacaggtccgaatatgggaggaaagtcgtctttcatccgccagatcgcactgcttgtggtcatggcacagataggctgctacatacctgcggactccgggtccaagctgagcattttcgactcgatccacacccgaatgggcgcacaagacgatattatcaagggagagtctacgtttcaggtggagctgaaggaatgcagcaccattttgaaagagtgcggaccacggtcgctggttctgatggacgaagtgggccgcggaaccagtaccatggacggcttcgccatagcgcattccattctgcggtaccttgttacagataggtcgccgtttgtactattcattacacactatcagaacctgaggtcgtttgagaggttcaaagaggtgaaaagttaccatatgggcatccaaaaagtggatgaagacatcgtcttcacatataagctatccgcaggatgctctgatcgttcatacggtatcaattgtgcaaagttggcaggactgcctaaacctgttcttgaatccgcacatcagaactcagtcaggttcgaaaatgactggagactcaaggaggcactgagtctggcacacaactttcgcactctcatagaaaataaagattatgccaagctgctggaactggcacaagatctggtgtgatcacattttttttgtagaaatgtcttggtgtcctcgtccaatcaggtagccatct

SEQ ID NO: 59 - DNA, 70 bp, Artificial Sequence
  Primer 40
ttatgccaagctgctggaactggcacaagatctggtgtgatcacattttttttgtagaaatgtcttggtg

SEQ ID NO: 60 - DNA, 70 bp, Artificial Sequence
  Primer 41
caaagtagtaaccaaagttggccatggaactggcaacttaccagtagtacagatgaacttcaaagtcaac

SEQ ID NO: 61 - DNA, 735 bp, Artificial Sequence
  PgapGFP1
ttatgccaagctgctggaactggcacaagatctggtgtgatcacattttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacaactagtatggtttctaagggtgaagagttgttcactggtgttgttccaatcttggttgagttggacggtgacgttaacggacacaagttctctgtttctggtgaaggtgagggtgacgctacttacggaaagttgactttgaagttcatctgtactactggtaagttgccagttccatggccaactttggttactactttg

SEQ ID NO: 62 - DNA, 70 bp, Artificial Sequence
  Primer 42
gtgagggtgacgctacttacggaaagttgactttgaagttcatctgtactactggtaagttgccagttcc

SEQ ID NO: 63 - DNA, 100 bp, Artificial Sequence
  Primer 43
caattcaatcagataattaataatatcaaagttctatcaaaatacatgtattatacccccgatgatgtacaacgcgttacttgtacaactcgtccatacc

SEQ ID NO: 64 - DNA, 696 bp, Artificial Sequence
  GFP2
gtgagggtgacgctacttacggaaagttgactttgaagttcatctgtactactggtaagttgccagttccatggccaactttggttactactttgacttacggtgttcagtgtttctccagatacccagaccacatgaagcagcacgatttcttcaagtctgctatgccagagggttacgttcaagagagaactatcttcttcaaggacgacggtaactacaagactagagctgaggttaagttcgagggtgacacattggttaacagaatcgagttgaagggtatcgacttcaaagaggacggaaacatcttgggtcacaagttggagtacaactacaactcccacaacgtttacatcatggctgacaagcagaagaacggtatcaaggttaacttcaagatcagacacaacatcgaggacggttccgttcaattggctgaccactaccaacagaacactccaattggtgacggtccagttttgttgccagacaaccactacttgtccactcaatccgctttgtccaaggacccaaacgagaagagagatcacatggttttgttggagttcgttactgctgctggtatcactttgggtatggacgagttgtacaagtaacgcgttgtacatcatcgggggtataatacatgtattttgatagaactttgatattattaattatctgattgaattg

SEQ ID NO: 65 - DNA, 24 bp, Artificial Sequence
  Primer 44
gcttactttcataattgcgactgg

SEQ ID NO: 66 - DNA, 76 bp, Artificial Sequence
  Primer 45
tgagcggataacaatttcacacaggaaacagctatgaccatgattacgccttttttgtagaaatgtcttggtgtcc

SEQ ID NO: 67 - DNA, 70 bp, Artificial Sequence
  Primer 46
ttctcgtaagtgcccaacttgaactgaggaacagtcatgtctaaggttacttgtacaactcgtccatacc

SEQ ID NO: 68 - DNA, 1309 bp, Artificial Sequence
  Fragment G1
tgagcggataacaatttcacacaggaaacagctatgaccatgattacgccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacaactagtatggtttctaagggtgaagagttgttcactggtgttgttccaatcttggttgagttggacggtgacgttaacggacacaagttctctgtttctggtgaaggtgagggtgacgctacttacggaaagttgactttgaagttcatctgtactactggtaagttgccagttccatggccaactttggttactactttgacttacggtgttcagtgtttctccagatacccagaccacatgaagcagcacgatttcttcaagtctgctatgccagagggttacgttcaagagagaactatcttcttcaaggacgacggtaactacaagactagagctgaggttaagttcgagggtgacacattggttaacagaatcgagttgaagggtatcgacttcaaagaggacggaaacatcttgggtcacaagttggagtacaactacaactcccacaacgtttacatcatggctgacaagcagaagaacggtatcaaggttaacttcaagatcagacacaacatcgaggacggttccgttcaattggctgaccactaccaacagaacactccaattggtgacggtccagttttgttgccagacaaccactacttgtccactcaatccgctttgtccaaggacccaaacgagaagagagatcacatggttttgttggagttcgttactgctgctggtatcactttgggtatggacgagttgtacaagtaaccttagacatgactgttcctcagttcaagttgggcacttacgagaa

SEQ ID NO: 69 - DNA, 29 bp, Artificial Sequence
  Primer 47
ccttagacatgactgttcctcagttcaag

SEQ ID NO: 70 - DNA, 21 bp, Artificial Sequence
  Primer 48
atcgcgattcttgggtgagga

SEQ ID NO: 71 - DNA, 584 bp, Artificial Sequence
  Fragment G2
ccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttagatcttctagagacaataagaagaaaaaaaaagaaaagcggtgggggagggattattaaataaggattatgtaaccccagggtaccgttctatacatatttaaggattatttaggacaatcgatgaaatcggcatcaaactggatgggagtatagtgtccggataatcggataaatcatcttgcgaggagccgcttggttggttggtgagaggagtgaaatatgtgtctcctcacccaagaatcgcgat

SEQ ID NO: 72 - DNA, 20 bp, Artificial Sequence
  Primer 49
atcagcaccctgtgggggac

SEQ ID NO: 73 - DNA, 22 bp, Artificial Sequence
  Primer 50
ggcgtaatcatggtcatagctg

SEQ ID NO: 74 - DNA, 4095 bp, Artificial Sequence
  Fragment G3
atcagcaccctgtgggggacactattggcctccctcccaaaccttcgatgtggtagtgctttattatattgattacattgattacatagctaaaccctgcctggttgcaagttgagctccgaattccaatattagtaaaatgcctgcaagataacctcggtatggcgtccgaccccgcttaattattttaactcctttccaacgaggacttcgtaatttttgattagggagttgagaaacggggggtcttgatacctcctcgatttcagatcccaccccctctcagtcccaagtgggacccccctcggccgtgaaatgcgcgcactttagtttttttcgcatgtaaacgccggtgtccgtcatctagagaattccccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatatggtttctatattatctttgtactaaagagcaattgataatgtgcgagaaaaactggtccttatatgccgtttgcagcactccctcccgaactttacgaaaagtcgtgcgccacctgattttcatcacgccaaaaacctacacgtatgactactccgggccagtgttcaccacgagctatatagtgttaattaattaccttattggttagctctgcatgtaagggtggtgtgagccgggaattgggtctactctagcgttcagtaaggtgatataaagctctgtagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 75 - DNA, 20 bp, Artificial Sequence
  Primer 51
ttcgtcaacctcttgcggag

SEQ ID NO: 76 - DNA, 29 bp, Artificial Sequence
  Primer 52
gctttatatatgggagaaagttaactacg

(19) United States
(12) Patent Application Publication
(10) Pub. No.: US 2020/0140873 A1
(43) Pub. Date: May 7, 2020

(54) METHOD FOR ASSEMBLING VECTORS EFFICIENTLY IN METHANOL-UTILIZING YEAST STRAIN

(71) Applicant: KANEKA CORPORATION, Osaka (JP)

(72) Inventors: Teruyuki Nishi, Hyogo (JP); Toru Watanabe, Hyogo (JP)

(73) Assignee: KANEKA CORPORATION, Osaka (JP)

(21) Appl. No.: 16/723,155

(22) Filed: Dec. 20, 2019

Related U.S. Application Data
(63) Continuation of application No. PCT/JP2018/023679, filed on Jun. 21, 2018.

(30) Foreign Application Priority Data
Jun. 23, 2017 (JP) 2017-122788
Dec. 22, 2017 (JP) 2017-246316

(51) Int. Cl. C12N 15/81

(52) U.S. Cl. CPC C12N 15/815

(57) ABSTRACT

A method for assembling two or more types of vectors includes introducing the two or more types of vectors into a methanol-utilizing yeast strain. The methanol-utilizing yeast strain comprises a DNL4 gene that is inactivated, and the two or more types of vectors are assembled in a transformant of the methanol-utilizing yeast strain.

### TECHNICAL FIELD

One or more embodiments of the present invention are related to a method for assembling vectors in a methanol-utilizing yeast strain in which the DNL4 gene associated with non-homologous end joining has been inactivated.

### BACKGROUND

In order to artificially engineer microorganisms such as yeast strains for industrial use, in recent years, a various techniques including integration of a gene fragment into the genome, development of an autonomously replicating vector, genome editing, and construction of long-chain artificial chromosomes have been attempted. In genome editing, such as genome integration, however, a target gene may not be integrated into a site of interest, and an unanticipated influence caused by a significantly changed genome structure is an issue of concern. While an autonomously replicating vector that involves a small risk in genome integration has been extensively used in procaryotic/eucaryotic organisms, such as E. coli or yeast strains, construction of, in particular, long-chain DNA, still remains difficult.

As a means of dissolving the problems described above, a method for assembling vectors in a budding yeast (Saccharomyces cerevisiae) using an autonomously replicating vector has been reported (Non-Patent Literature 1). Also, methods for assembling vectors in a type of methanol-utilizing yeast strain that has been used at the industrial level since early times; i.e., Pichia pastoris, using an autonomously replicating vector have been reported (Patent Literature 1 and Non-Patent Literatures 2 and 3).

### PATENT LITERATURES

### [Patent Literature 1]

WO 2017/055436

### Non-Patent Literatures

### [Non-Patent Literature 1]

Nucleic Acids Res. 2009 November; 37 (20): 6984-6990

### [Non-Patent Literature 2]

Protein Expr. Purif., 2011 May; 77 (1): 1-8

### [Non-Patent Literature 3]

Microb. Cell Fact., 2016 Aug. 11; 15 (1): 139

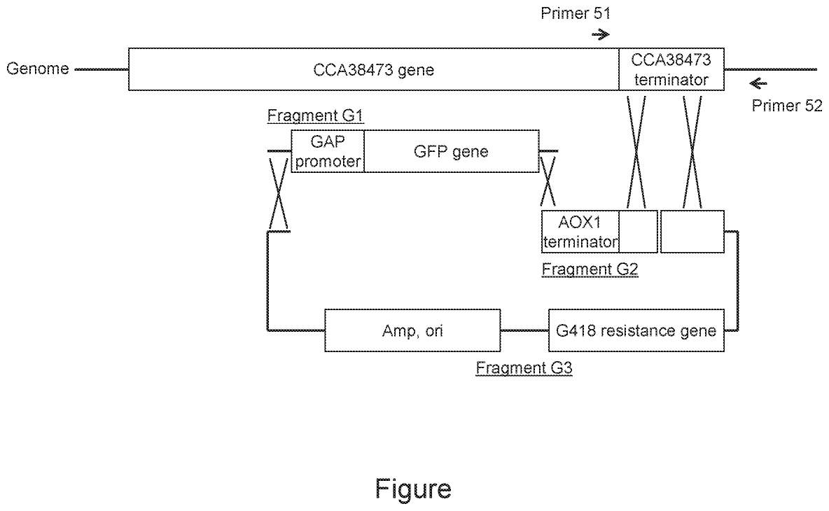

When assembling vectors in a yeast strain by the methods described in Patent Literature 1 and Non-Patent Literatures 2 and 3, however, other problems and complications, such as self-circularization of a vector caused by non-homologous end joining, low assembly efficiency, selection of pseudopositive strains, and fractional use of selection marker genes for suppressing the background pseudo-positive strains, arise. According to Patent Literature 1, in particular, self-circularization of a vector cannot be suppressed irrespective of the use of a yeast strain in which the KU70 gene associated with non-homologous end joining has been inactivated (Patent Literature 1; Example 9 and FIG. 27).

### SUMMARY

One or more embodiments of the present invention provide a novel means of completely suppressing self-circularization of a vector to suppress the background pseudo-positive strains at the time of transformation of a methanol-utilizing yeast strain and assembling vectors efficiently in a yeast strain.

The present inventors had conducted extensive analysis on the nucleotide sequence of chromosome DNA of a yeast strain belonging to the genus Komagataella to identify the DNL4 protein associated with non-homologous end joining. They also found that self-circularization of a vector could be completely suppressed by inactivating a gene encoding such protein. In addition, they found that vectors could be assembled efficiently in a yeast strain in which the gene was inactivated. This has led to the completion of one or more embodiments of the present invention.

Specifically, one or more embodiments of the present invention include the following.

(1) A method for assembling two or more types of vectors by a transformation method comprising a step of introducing the two or more types of vectors into a methanol-utilizing yeast strain in which the DNL4 gene has been inactivated.
(2) The method according to (1), wherein the DNL4 gene is any of the genes (a) to (d) below:

(a) a gene comprising the nucleotide sequence as shown in SEQ ID NO: 25;

(b) a gene comprising a nucleotide sequence of a nucleic acid hybridizing under stringent conditions to a nucleic acid comprising a nucleotide sequence complementary to the nucleotide sequence as shown in SEQ ID NO: 25;

(c) gene comprising a nucleotide sequence having 85% or higher sequence identity to the nucleotide sequence as shown in SEQ ID NO: 25; and

(d) a gene encoding an amino acid sequence having 85% or higher sequence identity to the amino acid sequence as shown in SEQ ID NO: 24.

(3) The method according to (1) or (2), wherein the methanol-utilizing yeast strain is a yeast strain belonging to the genus Komagataella or a yeast strain belonging to the genus Ogataea.
(4) The method according to any of (1) to (3), wherein the two or more types of vectors each comprise a nucleotide sequence having 85% or higher sequence identity to one another.
(5) The method according to any of (1) to (4), wherein at least one of the two or more types of vectors comprises an autonomously replicating sequence (ARS).
(6) The method according to any of (1) to (5), wherein at least one of the two or more types of vectors is an autonomously replicating vector.
(7) The method according to (6), wherein the autonomously replicating vector further comprises an autonomously replicating sequence (ARS) and/or a centromeric DNA sequence derived from a yeast strain belonging to the genus Komagataella or a yeast strain belonging to the genus Ogataea.
(8) A method for producing assembled vectors comprising the method according to any of (1) to (7).
(9) Assembled vectors obtained by the method according to any of (1) to (8).
(10) A method for producing a transformant comprising a step of transformation with the assembled vectors obtained by the method according to any of (1) to (8).
(11) A transformant transformed with the assembled vectors according to (9).

The present specification encompasses the content disclosed in JP Patent Application Nos. 2017-122788 and 2017-246316 to which present application claims priority.

One or more embodiments of the present invention can suppress development of background pseudo-positive strains in transformation of a methanol-utilizing yeast strain by completely suppressing self-circularization of a vector. In addition, one or more embodiments of the present invention provide a novel means for assembling vectors efficiently in a yeast strain.

### BRIEF DESCRIPTION OF THE DRAWINGS

The FIGURE schematically shows the constitutions of vector fragments used for genome integration used in Production Example 7, Comparative Example 4, and Example 5.

### DETAILED DESCRIPTION OF EMBODIMENT

Hereinafter, one or more embodiments of the present invention are described in detail.

The term “non-homologous end joining” used herein refers to a mechanism in which ends of the cleaved DNA double strands are bound to each other. For example, KU70 and KU80 dimers recognize the ends and a DNL4-LIF1 complex as a ligase binds the ends to each other.

A gene associated with non-homologous end joining is a gene of a protein associated with the mechanism described above, and examples thereof include KU70, KU80, DNL4, and LIF1 genes. In the case of the Komagataella pastoris strain ATCC76273, for example, the KU70 gene is represented by a nucleotide sequence encoding a polypeptide as shown in Accession No. CCA39840 (KU70), the KU80 gene is represented by a nucleotide sequence encoding a polypeptide as shown in Accession No. CCA40385 (KU80), and the DNL4 gene is represented by a nucleotide sequence encoding a polypeptide as shown in Accession No. CCA39424 (DNL4).

According to one or more embodiments of the present invention, the DNL4 gene is any of the genes (a) to (d) below:

(a) a gene comprising the nucleotide sequence as shown in SEQ ID NO: 25;

(b) a gene comprising a nucleotide sequence of a nucleic acid hybridizing under stringent conditions to a nucleic acid comprising a nucleotide sequence complementary to the nucleotide sequence as shown in SEQ ID NO: 25;

(c) a gene comprising a nucleotide sequence having 85% or higher sequence identity to the nucleotide sequence as shown in SEQ ID NO: 25; and

(d) a gene encoding an amino acid sequence having 85% or higher sequence identity to the amino acid sequence as shown in SEQ ID NO: 24.

When two nucleic acids hybridize to each other under stringent conditions in one or more embodiments of the present invention, for example, nucleic acid Y is considered as “the nucleic acid that hybridizes under stringent conditions to the nucleic acid X” when the nucleic acid Y can be obtained as the nucleic acid bound on a filter by using the filter with an immobilized nucleic acid X, hybridizing it to a nucleic acid Y exhibiting sequence identity of 85% or higher at 65° C. in the presence of 0.7 to 1.0 M NaCl, and then washing the filter under the condition of 65° C. using a 2-fold SSC solution (the composition of 1-fold SSC solution consists of 150 mM sodium chloride and 15 mM sodium citrate). Alternatively, the nucleic acid X and the nucleic acid Y can be said to “hybridize to each other under stringent conditions.” As the concentration of the SSC solution is lowered, hybridization of nucleic acids with higher sequence identity can be expected. Accordingly, in one or more embodiments, the nucleic acid Y can be obtained in the form of the nucleic acid bound on a filter when the filter is washed preferably at 65° C. using a 1-fold SSC solution, more preferably at 65° C. using a 0.5-fold SSC solution, still more preferably at 65° C. using a 0.2-fold SSC solution, and further preferably at 65° C. using a 0.1-fold SSC solution. In addition, hybridization of nucleic acids with higher sequence identity can be expected as temperature increases. Accordingly, in one or more embodiments, the nucleic acid Y can be obtained in the form of the nucleic acid bound on a filter when the filter is washed preferably at 70° C. using a 2-fold SSC solution, more preferably at 75° C. using a 2-fold SSC solution, still more preferably at 80° C. using a 2-fold SSC solution, and further preferably at 85° C. using a 2-fold SSC solution. The standard nucleic acid X may be a colony- or plaque-derived nucleic acid X.

The sequence identity of a nucleotide sequence and an amino acid sequence in one or more embodiments of the present invention can be determined by a method or with the use of sequence analysis software known to a person skilled in the art. Examples include the Blastn program and Blastp program of BLAST algorithm, and Fasta program of FASTA algorithm. In one or more embodiments of the present invention, the “sequence identity” of a certain nucleotide sequence to be evaluated to a nucleotide sequence X is a value shown in % of the frequency with which the same nucleotides appear in the same sites of the nucleotide sequences including the gap portions, when the nucleotide sequence X and the nucleotide sequence to be evaluated are aligned and gaps are introduced as needed to achieve the highest nucleotide alignment between both sequences. In one or more embodiments of the present invention, the “sequence identity” of a certain amino acid sequence to be evaluated to an amino acid sequence X is a value shown in % of the frequency with which the same amino acid appears in the same site of the amino acid sequences including the gap portions, when the amino acid sequence X and the amino acid sequence to be evaluated are aligned and gaps are introduced as needed to achieve the highest amino acid alignment between both sequences.

In one or more embodiments, the sequence identity between the gene indicated above and the nucleotide sequence as shown in SEQ ID NO: 25 is 85% or higher, preferably 90% or higher, more preferably 95% or higher, further preferably 96% or higher, particularly preferably 97% or higher, 98% or higher, and most preferably 99% or higher.

According to one or more embodiments of the present invention, a “nucleic acid” may also be referred to as a “polynucleotide,” it refers to DNA or RNA, and it typically refers to DNA. Double-stranded or single-stranded DNA may be used, DNA comprising a given nucleotide sequence may be double-stranded DNA comprising the given nucleotide sequence in one strand, single-stranded DNA comprising the given nucleotide sequence (a sense strand), or single-stranded DNA comprising a sequence complementary to the given nucleotide sequence (an anti-sense strand).

The term “a nucleotide sequence encoding an amino acid sequence” used herein refers to a nucleotide sequence designed based on a codon table to a polypeptide consisting of an amino acid sequence, and such nucleotide sequence produces a polypeptide by transcription and translation.

In one or more embodiments of the present invention, the “polypeptide” comprises 2 or more amino acids peptide-bonded to one another, and it encompasses a short-chain substance referred to as a peptide or oligopeptide in addition to a protein.

In one or more embodiments of the present invention, the “gene” encompasses DNA and its transcript, mRNA, unless otherwise specified. It is typically DNA, and in one or more embodiments, it is particularly preferably DNA included in a host chromosome. In an embodiment in which a gene is mRNA, T (thymine) in a given nucleotide sequence (e.g., SEQ ID NO: 25) may be read as U (uracil). The term “gene” used herein refers to any of a regulatory region, a coding region, an exon, and an intron, unless otherwise specified.

When a gene is “inactivated” in one or more embodiments of the present invention, gene functions are lost or attenuated. When a gene is “inactivated,” the expression level of a transcript of such gene, mRNA, or a translation product, polypeptide, is low, or mRNA or a protein does not normally function. The mRNA expression level can be quantified by, for example, real-time PCR, RNA-Seq, Northern hybridization, or DNA array-based hybridization. The polypeptide expression level can be quantified with the use of, for example, an antibody that recognizes a polypeptide or a dye compound that can bind to a polypeptide. In addition to the quantification methods mentioned above, a conventional technique employed by a person skilled in the art may be employed herein.

A gene can be inactivated by, for example, DNA mutation using an agent or ultraviolet rays, site-directed mutagenesis using PCR, RNAi, a protease, or homologous recombination. When a gene is to be inactivated, a nucleotide sequence in an ORF of the target gene may be modified (via deletion, substitution, addition, or insertion) and/or a nucleotide sequence in a region that regulates initiation or termination of transcription, such as a promoter region, an enhancer region, or a terminator region, may be modified (via deletion, substitution, addition, or insertion). A site to be subjected to deletion, substitution, addition, or insertion or a nucleotide sequence to be subjected to deletion, substitution, addition, or insertion is not particularly limited, provided that normal functions of the target gene can be deleted. A gene to be inactivated may be located inside or outside the chromosome of a methanol-utilizing yeast strain. The gene located inside the chromosome may be preferably inactivated. In one or more embodiments, the DNL4 gene can be inactivated by deleting at least a part of the gene coding region in the host chromosome (e.g., a nucleotide sequence region comprising the number of nucleotides that is 50% or more, preferably 60% or more, more preferably 70% or more, still more preferably 80% or more, further preferably 90% or more, and most preferably 100% relative to the number of nucleotides constituting the nucleotide sequence of the coding region).

In one or more embodiments of the present invention, “modification of a nucleotide sequence” can be carried out by a technique, such as gene insertion via homologous recombination or site-directed mutagenesis. For example, an upstream promoter may be substituted with a promoter with lower activity, a codon may be modified into another codon that is not suitable for a methanol-utilizing yeast strain, or a vector comprising an upstream sequence of the gene to be deleted, a selection marker gene sequence, and a downstream sequence of the gene to be deleted ligated thereto may be introduced.

In one or more embodiments of the present invention, an extent of lowering in the expression level is not particularly limited, and an extent of lowering is preferably 5% or more, 10% or more, 20% or more, 30% or more, 40% or more, 50% or more, 60% or more, 70% or more, 80% or more, 90% or more, or 95% or more. It is well-known to a person skilled in the art that the gene can be inactivated even if the expression level is not lowered, provided that normal functions of a gene product can be deleted as described above. Thus, the extent of lowering in the expression level is merely a criterion for inactivation.

The number of inactivated DNL4 genes is not particularly limited. In the methanol-utilizing yeast strain according to one or more embodiments of the present invention, other genes associated with non-homologous end joining may further be inactivated. For example, dual inactivation of the KU70 gene and DNL4 gene, KU80 gene and DNL4 gene, or DNL4 gene and LIF1 gene, triple inactivation of KU70 gene, KU80 gene, and DNL4 gene, KU70 gene, DNL4 gene, and LIF1 gene, or KU80 gene, DNL4 gene, and LIF1 gene, or quadruple inactivation of KU70 gene, KU80 gene. DNL4 gene, and LIF1 gene may be employed.

The “gene comprising the nucleotide sequence as shown in SEQ ID NO: 25” was found as a result of extensive analysis of the nucleotide sequences of 4 chromosome DNAs of Komagataella pastoris (the strain ATCC76273: Accession Nos. FR839628 to FR839631 (J. Biotechnol., 154 (4), 312-320, 2011); and the strain GS115: Accession Nos. FN392319 to FN392322 (Nat. Biotechnol., 27 (6), 561-566, 2009)). Specifically, the present inventors searched for a polypeptide capable of suppressing self-circularization of a vector via inactivation and a polynucleotide comprising a nucleotide sequence encoding the amino acid sequence of the polypeptide. As a result, they found a polypeptide comprising an amino acid sequence (Accession No. CCA39424) as shown in SEQ ID NO: 24 (DNL4) and a polynucleotide comprising a nucleotide sequence as shown in SEQ ID NO: 25 encoding such polypeptide in the strain ATCC76273. In the examples described below, the present inventors completed a method that enables efficient vector assembly in yeast with the use of, as a host cell, a methanol-utilizing yeast strain in which a gene comprising the nucleotide sequence has been inactivated.

In a method for inactivating the gene comprising the nucleotide sequence as shown in SEQ ID NO: 25 (i.e., the DNL4 gene), for example, a nucleotide sequence exhibiting 100% sequence identity to a 1,000-bp promoter located upstream of the gene comprising the nucleotide sequence as shown in SEQ ID NO: 25 (e.g., the nucleotide sequence as shown in SEQ ID NO: 1) and a nucleotide sequence exhibiting 100% sequence identity to a 1,007-bp terminator located downstream of the gene comprising the nucleotide sequence as shown in SEQ ID NO: 25 (e.g., the nucleotide sequence as shown in SEQ ID NO: 2) are prepared as PCR products with the use of primers 1 to 4, as described in the examples below. A vector comprising the PCR products is transformed into a yeast strain belonging to the genus Komagataella, and homologous recombination may be carried out with the use of the selected strain. Thus, the gene of interest can be deleted.

The “gene encoding an amino acid sequence having 85% or higher sequence identity to the amino acid sequence as shown in SEQ ID NO: 24” comprises a nucleotide sequence designed with reference to a codon table based on a polypeptide comprising an amino acid sequence having 85% or higher sequence identity to the amino acid sequence as shown in SEQ ID NO: 24. An example of such gene is the gene as shown in SEQ ID NO: 25. By inactivating such gene, self-circularization of a vector can be suppressed, and vectors can be assembled efficiently in a yeast strain. In one or more embodiments, the sequence identity is 85% or higher, preferably 90% or higher, more preferably 95% or higher, further preferably 96% or higher, particularly preferably 97% or higher, 98% or higher, and most preferably 99% or higher.

A methanol-utilizing yeast strain according to one or more embodiments of the present invention in which the DNL4 gene has been inactivated may be preferably obtained by transforming a methanol-utilizing yeast strain with a vector comprising at least one of a nucleotide sequence having a homologous region to that of the DNL4 gene, a nucleotide sequence comprising a homologous region to that of the DNL4 gene promoter, and a nucleotide sequence comprising a homologous region to a DNL4 gene terminator.

The methanol-utilizing yeast strain according to one or more embodiments of the present invention in which the DNL4 gene has been inactivated may be preferably obtained by transforming a methanol-utilizing yeast strain with a vector comprising at least one of, for example, a nucleotide sequence having a homologous region to that of any of the genes (a) to (d) above, a nucleotide sequence comprising a homologous region to that of the promoter of any of the genes (a) to (d) above, and a nucleotide sequence comprising a homologous region to that of the terminator of any of the genes (a) to (d) above.

In one or more embodiments of the present invention, the DNL4 gene in the chromosome of the methanol-utilizing yeast strain, such as any of the genes (a) to (d) above, can be inactivated via transformation.

In one or more embodiments in which the DNL4 gene, which is any of the genes (a) to (d) above, is inactivated, a DNA fragment 1 comprising a nucleotide sequence having 85% or higher sequence identity to a partial nucleotide sequence of the nucleotide sequence of the promoter located upstream of the gene in the host chromosome (i.e., a partial nucleotide sequence 1) and a DNA fragment 2 comprising a nucleotide sequence having 85% or higher sequence identity to a partial nucleotide sequence of the nucleotide sequence of the terminator located downstream of the gene (i.e., a partial nucleotide sequence 2) are ligated to each other, so that the DNA fragment 1 is located upstream and the DNA fragment 2 is located downstream, so as to construct a vector. The resulting vector is transformed into a host, and the gene of interest is deleted via homologous recombination. The vector may be preferably a linear vector prepared by cleaving a cyclic vector comprising the DNA fragment 1 and the DNA fragment 2 at the restriction enzyme recognition site inside the DNA fragment 1 or the DNA fragment 2 and linearizing the resultant. An example of the nucleotide sequence of the promoter is the nucleotide sequence as shown in SEQ ID NO: 1. An example of the nucleotide sequence of the terminator is the nucleotide sequence as shown in SEQ ID NO: 2. The length of the partial nucleotide sequence 1 and that of the partial nucleotide sequence 2 are not particularly limited, as long as homologous recombination can be implemented. In one or more embodiments, the length is preferably 100 bp or longer, 200 bp or longer, 300 bp or longer, 400 bp or longer, 500 bp or longer, 600 bp or longer, 700 bp or longer, 800 bp or longer, 900 bp or longer, or 1,000 bp or longer. In one or more embodiments, the sequence identity of 85% or higher mentioned above is preferably 90% or higher, more preferably 95% or higher, more preferably 96% or higher, more preferably 97% or higher, more preferably 98% or higher, and more preferably 99% or higher.

In one or more embodiments of the present invention, the methanol-utilizing yeast is defined as a yeast which can be cultured utilizing methanol as an only carbon source, but yeast which was originally a methanol-utilizing yeast but lost the methanol-utilizing ability due to an artificial modification or mutation is also within the scope of the methanol-utilizing yeast of the present invention.

Examples of the methanol-utilizing yeast strains include yeast strains belonging to the genus Pichia, the genus Ogataea, the genus Candida, the genus Torulopsis, and the genus Komagataella. In one or more embodiments, preferable examples include Pichia methanolica in the genus Pichia, Ogataea angusta, Ogataea polymorpha, Ogataea parapolymorpha, and Ogataea minuta in the genus Ogataea, Candida boidinii in the genus Candida, and Komagataella pastoris and Komagataella phaffii in the genus Komagataella.

In one or more embodiments, among the methanol-utilizing yeast strains described above, yeast strains belonging to the genus Komagataella or yeast strains belonging to the genus Ogataea are particularly preferable.

In one or more embodiments, as yeast strains belonging to the genus Komagataella, Komagataella pastoris and Komagataella phaffii are preferable. Both Komagataella pastoris and Komagataella phaffii are also referred to as “Pichia pastoris.”

Specific examples of strains that can be used as hosts include Komagataella pastoris ATCC76273 (Y-11430, CBS7435) and Komagataella pastoris X-33. These strains are available from American Type Culture Collection or Thermo Fisher Scientific, Inc.

In one or more embodiments, as yeast strains belonging to the genus Ogataea, Ogataea angusta, Ogataea polymorpha, and Ogataea parapolymorpha are preferable. These 3 strains are closely related to each other and are also referred to as “Hansenula polymorpha” or “Pichia angusta.”

Specific examples of strains that can be used include Ogataea angusta NCYC495 (ATCC14754). Ogataea polymorpha 8V (ATCC34438), and Ogataea parapolymorpha DL-1 (ATCC26012). These strains are available from, for example, American Type Culture Collection.

In one or more embodiments of the present invention, in addition, derivative strains from the yeast strains belonging to the genus Komagataella or the genus Ogataea can also be used, an example of a histidine-auxotrophic yeast strain is the Komagataella pastoris GS115 strain (available from Thermo Fisher Scientific, Inc.), and examples of a leucine-auxotrophic yeast strains include NCYC495-derived BY4329, 8V-derived BY5242, and DL-1-derived BY5243 (these can be distributed from National BioResource Project). In one or more embodiments of the present invention, derivative strains from these strains can also be used.

The vector according to one or more embodiments of the present invention (at least one of the two or more types of vectors used in the present invention and/or a vector formed of two or more types of vectors assembled to each other) can be a cyclic vector, a linear vector, a plasmid vector, or an artificial chromosome vector.

The “vector” in one or more embodiments of the present invention is a nucleic acid molecule that is artificially constructed. Nucleic acid molecules constituting the vector according to one or more embodiments of the present invention (the two or more types of vectors used in one or more embodiments of the present invention and/or a vector formed of two or more types of vectors assembled to each other) is generally DNA, and it may be preferably double-stranded DNA. A cyclic or linear DNA may be used. At least one of the two or more types of vectors and a vector formed of two or more types of vectors assembled to each other used in one or more embodiments of the present invention can generally comprise a cloning site including 1 or more restriction enzyme recognition sites, an overlapping region for utilizing an In-Fusion cloning system of Clontech Laboratories, Inc. or a Gibson Assembly system of New England Biolabs, a nucleotide sequence of an endogenous gene, a nucleotide sequence encoding a polypeptide comprising the amino acid sequence of the target protein, and a nucleotide sequence of a selectable marker gene (e.g., an auxotrophic complementary gene and a drug resistance gene). Examples of linear vectors include auxotrophic complementary genes, such as URA3 gene, LEU2 gene, ADE1 gene, HIS4 gene, and ARG4 gene, PCR products comprising nucleotide sequences of drug resistant genes, such as G418 resistance gene. Zeocin (tradename) resistance gene, hygromycin resistance gene, Clone NAT resistance gene, and blasticidin S resistance gene, and linear vectors prepared by cleaving a cyclic or plasmid vector with an adequate restriction enzyme. Examples of plasmid vectors that can be used include YEp vector, YRp vector, YCp vector, pPICHOLI (http://www.mobitec.com/cms/prxoducts/bio/04_vector_sys/p_picholi_shuttle_vector.html), pHIP (Journal of General Microbiology, 1992, 138, 2405-2416. Chromosomal targeting of replicating plasmids in the yeast Hansenula polymorpha), pHRP (see the documents referred concerning pHIP above), pHARS (Molecular and General Genetics MGG February 1986, Volume 202, Issue 2, pp. 302-308, Transformation of the methylotrophic yeast Hansenula polymorpha by autonomous replication and integration vectors), E. coli-derived plasmid vectors (pUC18, pUC19, pBR322, pBluescript, and pQE), and Bacillus subtilis-derived plasmid vectors (pHY300PLK and pMTLBS72). In general, examples of artificial chromosome vectors include artificial chromosome vectors comprising centromeric DNA sequence, telomeric DNA sequence, and autonomously replicating sequence (ARS). In the case of yeast, an example is an yeast artificial chromosome (YAC) vector. Such vectors have been developed in, for example, Saccharomyces cerevisiae or Schizosaccharomyces pombe. In the case of Komagataella pastoris, an artificial chromosome vector can be constructed with the use of the centromeric DNA sequence described in WO 2016/088824.

In one or more embodiments of the present invention, the term “transformation” refers to introduction of the vector into the methanol-utilizing yeast strain. Introduction of a vector into a methanol-utilizing yeast strain; i.e., transformation, can be adequately carried out via a known technique, and examples thereof include, but are not particularly limited to, the electroporation method, the lithium acetate method, and the spheroplast method. For example, the electroporation method described in High efficiency transformation by electroporation of Pichia pastoris pretreated with lithium acetate and dithiothreitol (Biotechniques, January 2004; 36 (1): 152-4) is a common transformation method for Komagataella pastoris.

When a methanol-utilizing yeast strain is transformed with a vector in one or more embodiments of the present invention, a selectable marker gene such as an auxotrophic complementary gene or a drug resistance gene may be preferably used. A selectable marker is not particularly limited. In the case of a methanol-utilizing yeast strain, an auxotrophic complementary gene such as URA3 gene, LEU2 gene, ADE1 gene. HIS4 gene, or ARG4 gene enables selection of a transformant by the recovery of a prototrophic phenotype in auxotrophic strains of uracil, leucine, adenine, histidine, and arginine, respectively. When a vector comprising a drug resistance gene such as G418 resistance gene, Zeocin (tradename) resistance gene, hyglomycin resistance gene, Clone NAT resistance gene, or blasticidin S resistance gene is used, alternatively, the transformant can be selected based on the drug resistance on the relevant medium containing G418, Zeocin (tradename), and hyglomycin, Clone NAT, or blasticidin S, respectively. It is not possible to use an auxotrophic selectable marker used for preparing a transformant when such selectable marker is not disrupted in the host. In such a case, the selectable marker may be disrupted in the host, and a method known to a person skilled in the art can be employed.

According to the method of transformation according to one or more embodiments of the present invention, vectors can be efficiently assembled in yeast. Thus, the method comprises a step of introducing two or more types of vectors. In one or more embodiments, two or more types of vectors are introduced successively or simultaneously. For example, competent cells are mixed with two or more types of vectors before the process of transformation as performed in the present examples.

When transformation is carried out with the use of two or more types of vectors in one or more embodiments of the present invention, it may be preferable that vectors be constructed to comprise nucleotide sequences exhibiting sequence identity to each other as described in the examples below. It may be more preferable that each of two or more types of vectors comprise, as a partial nucleotide sequence, a nucleotide sequence exhibiting sequence identity to that of at least an other vector. In the nucleotide sequences, the number of sites exhibiting sequence identity may be 1, or it may be 2 or more.

In the “nucleotide sequences exhibiting sequence identity to each other” according to one or more embodiments of the present invention, at least a part of one nucleotide sequence is identical to that of the other nucleotide sequence. In one or more embodiments, such nucleotide sequences exhibit sequence identity of preferably 85% or higher, more preferably 90% or higher, further preferably 95% or higher, and most preferably 100%. The length of such nucleotide sequences exhibiting sequence identity is not particularly limited, provided that it is 20 bp or longer. Thus, two vectors comprising such nucleotide sequences can be assembled. Specifically, the length of nucleotide sequences may be preferably 20 bp or longer, 30 bp or longer, 40 bp or longer, 50 bp or longer, 60 bp or longer, 70 bp or longer, 80 bp or longer, 90 bp or longer, 100 bp or longer, 200 bp or longer, 300 bp or longer, 400 bp or longer, 500 bp or longer, or 1,000 bp or longer.

When transformation is carried out with the use of two or more types of vectors in one or more embodiments of the present invention, the molar ratio thereof is not particularly limited. When two types of vectors are used, for example, the molar ratio may be preferably 1:1, 1:1 or more, 1:2 or more, 1:3 or more, 1:4 or more, 1:5 or more, 1:10 or more, 1:20 or more, 1:30 or more, 1:40 or more, or 1:50 or more.

The term “assembly” used in one or more embodiments of the present invention refers to a mechanism in which two or more types of vectors introduced into a yeast strain are ligated to each other with the aid of nucleotide sequences exhibiting sequence identity to each other.

According to one or more embodiments, each of two or more types of vectors comprises, as a partial nucleotide sequence, a nucleotide sequence exhibiting sequence identity to that of at least an other vector, the two or more types of vectors are assembled to each other via homologous recombination between such partial nucleotide sequences, and one linear or cyclic vector is constructed as a consequence. The one linear vector may be integrated into the host genome DNA. In such a case, one of the two or more types of vectors further may comprise, as a partial nucleotide sequence, a nucleotide sequence exhibiting sequence identity (preferably 85% or higher, more preferably 90% or higher, further preferably 95% or higher, and most preferably 100%) to the nucleotide sequence located upstream of the site where the vector has been inserted into the host genome DNA. The other of the two or more types of vectors may further comprise, as a partial nucleotide sequence, a nucleotide sequence exhibiting sequence identity (preferably 85% or higher, more preferably 90% or higher, further preferably 95% or higher, and most preferably 100%) to the nucleotide sequence located downstream of the site where the vector has been inserted into the host genome DNA.

The “host cell” in one or more embodiments of the present invention refers to a cell to be transformed by introducing a vector thereinto. A host cell after transformation is occasionally referred to as the “transformant” herein. The cell used as the host is not particularly limited, as long as it is a methanol-utilizing yeast cell into which a vector can be introduced.

The vector according to one or more embodiments of the present invention (at least one of the two or more types of vectors used in the present invention and/or a vector formed of two or more types of vectors assembled to each other) can comprise an autonomously replicating sequence (ARS). In one or more embodiments of the present invention, ARS is a replication origin in methanol-utilizing yeast strains, such as prokaryotes (e.g., E. coli, bacteria, actinomycete, Eubacteria, archaebacteria, or blue-green algae), viruses (e.g., DNA viruses or RNA viruses), and eukaryotes (e.g., fungi, algae, protozoan, yeast, plants, animals, birds, fowls, mammals, humans, or mice), preferably in eukaryotes, and more preferably in yeast, such as Komagataella pastoris. It is a region in a nucleotide sequence where replication is initiated. The vector according to one or more embodiments of the present invention may comprise two or more ARSs of, for example, different yeast species. An example of ARS that can be within the scope of the vector according to one or more embodiments of the present invention is a centromeric DNA sequence comprising ARS in Komagataella pastoris comprising the nucleotide sequence as shown in SEQ ID NO: 6 described in the examples below. This sequence is a centromeric DNA sequence of chromosome DNA 2 of Komagataella pastoris (Accession No. FR839629).

Another example of ARS that can be within the scope of the vector according to one or more embodiments of the present invention (at least one of the two or more types of vectors used in one or more embodiments of the present invention and/or a vector formed of two or more types of vectors assembled to each other) is PARS1, which is ARS in Komagataella pastoris comprising the nucleotide sequence as shown in SEQ ID NO: 37, as described in the examples below. This sequence is a 164-bp ARS located in a region between position 1980709 and position 1980872 of chromosome DNA 2 of Komagataella pastoris (Accession No. FR839629).

The vector according to one or more embodiments of the present invention (at least one of the two or more types of vectors used in one or more embodiments of the present invention and/or a vector formed of two or more types of vectors assembled to each other) may not comprise an autonomously replicating sequence (ARS). For example, vectors assembled in yeast may be integrated into the host chromosome. According to one or more embodiments, two or more types of vectors are each a nucleic acid fragment incapable of autonomous replication, and a vector formed of the two or more types of vectors assembled to each other is integrated into the host genome DNA. According to one or more embodiments, two or more types of vectors are each a nucleic acid fragment incapable of autonomous replication, and the two or more types of vectors are integrated into the host genome DNA via homologous recombination in the host genome DNA region exhibiting sequence identity to the upper terminal and/or lower terminal sequence(s) of the vector formed of the two or more types of vectors assembled to each other.

The vector according to one or more embodiments of the present invention (at least one of the two or more types of vectors used in the present invention and/or a vector formed of two or more types of vectors assembled to each other) may be preferably an autonomously replicating vector. When the vector according to one or more embodiments of the present invention is an autonomously replicating vector, the host cell can be modified without changing the genome sequence or genome structure of the host cell. According to one or more embodiments, one of the two or more types of vectors is an autonomously replicating vector, the other of the two or more types of vectors is a nucleic acid fragment incapable of autonomous replication by itself, and a vector formed of the two or more types of vectors assembled to each other is an autonomously replicating vector.

In one or more embodiments of the present invention, an autonomously replicating vector is replicated independently of a host chromosome, and such vector is replicated in the host cell without being integrated into the host chromosome.

The vector according to one or more embodiments of the present invention can be used in the form of an autonomously replicating vector in a methanol-utilizing yeast strain, such as a yeast strain belonging to the genus Komagataella or a yeast strain belonging to the genus Ogataea, and preferably in Komagataella pastoris. Specifically, the autonomously replicating vector may preferably comprise ARS and/or a centromeric DNA sequence derived from a methanol-utilizing yeast strain, and in one or more embodiments, it more preferably comprises ARS and/or a centromeric DNA sequence derived from a yeast strain belonging to the genus Komagataella or a yeast strain belonging to the genus Ogataea.

The vector according to one or more embodiments of the present invention (at least one of the two or more types of vectors used in the present invention and/or a vector formed of two or more types of vectors assembled to each other) may be preferably an autonomously replicating vector that is capable of autonomous replication in host cells of a plurality of organism species. An example of such autonomously replicating vector is a vector comprising ARS and/or a centromeric DNA sequence derived from the yeast strain belonging to the genus Komagataella or the yeast strain belonging to the genus Ogataea and ARS and/or a centromeric DNA sequence derived from an organism species other than the organism species from which the aforementioned ARS and/or centromeric DNA sequence are/is derived. Specific examples of means that can be adopted to make the vector according to one or more embodiments of the present invention to autonomously replicate in hosts of other species or genera, as well as in Komagataella pastoris include: a means of cloning a centromeric DNA sequence of the human chromosome into a vector comprising a centromeric DNA sequence including ARS in Komagataella pastoris comprising the nucleotide sequence as shown in SEQ ID NO: 6 to prepare a human artificial chromosome: a means of cloning ARS or a centromeric DNA sequence of a yeast strain belonging to the genus Ogataea into a vector comprising the nucleotide sequence as shown in SEQ ID NO: 6 to prepare an autonomously replicating vector that can be used in the both genera; a means of cloning ARS or a centromeric DNA sequence of a budding yeast (Saccharomyces cerevisiae) or a fission yeast (Schizosaccharomyces pombe) into the vector comprising the nucleotide sequence as shown in SEQ ID NO: 6 to prepare an autonomously replicating vector that can be used in both the genera; and a means of cloning genes encoding proteins constituting the centromere of Komagataella pastoris into a vector comprising the nucleotide sequence as shown in SEQ ID NO: 6 to prepare an autonomously replicating vector in other genera. As described in the examples, the vector can be used in combination with an autonomously replicating vector in E. coli.

In one or more embodiments of the present invention, a centromeric DNA sequence is a nucleotide sequence that forms a structure referred to as a “kinetochore” to which a spindle binds. In humans, for example, it is a region where a long arm of the chromosome intersects with a short arm thereof, and it is located substantially in the middle of the chromosome. This region is accordingly referred to as a centromeric region.

Hereafter, one or more embodiments of the present invention are described in detail with reference to Production Examples, Comparative Examples, and Examples, although the present invention is not limited to these examples.

### Production Example 1: Preparation of Various Genes Used for Preparing Vectors

A detailed engineering method and the like related to recombinant DNA technology used in the Examples below are described in the following books: Molecular Cloning 2nd Edition (Cold Spring Harbor Laboratory Press, 1989) and Current Protocols in Molecular Biology (Greene Publishing Associates and Wiley-Interscience).

In the Examples below, a plasmid used for transforming a yeast was prepared by introducing a constructed vector into an Escherichia coli (E. coli) HST08 competent cells (Takara Bio Inc.), culturing the obtained transformants to amplify the plasmid. Preparation of the plasmid from the strain carrying the plasmid was performed by using FastGene Plasmid Mini Kit (NIPPON Genetics Co., Ltd.).

A DNL4 promoter (SEQ ID NO: 1), a DNL4 terminator (SEQ ID NO: 2), a promoter-regulated ADE1 gene sequence (SEQ ID NO: 3), a GAP promoter (SEQ ID NO: 4), a GAP1 terminator (SEQ ID NO: 5), a centromeric DNA sequence (SEQ ID NO: 6), and PARS1 (SEQ ID NO: 37) were used for vector construction. These sequences were prepared by PCR using, as a template, a mixture of chromosome DNA from the Komagataella pastoris strain ATCC76273 (the nucleotide sequences thereof are described in EMBL (The European Molecular Biology Laboratory) Accession Nos. FR839628 to FR839631). The DNL4 promoter was prepared by PCR using primer 1 (SEQ ID NO: 7) and primer 2 (SEQ ID NO: 8). The DNL4 terminator was prepared by PCR using primer 3 (SEQ ID NO: 9) and primer 4 (SEQ ID NO: 10). The promoter-regulated ADE1 gene sequence was prepared by PCR using primer 5 (SEQ ID NO: 11) and primer 6 (SEQ ID NO: 12). The GAP promoter was prepared by PCR using primer 7 (SEQ ID NO: 13) and primer 8 (SEQ ID NO: 14). The GAP1 terminator was prepared by PCR using primer 9 (SEQ ID NO: 15) and primer 10 (SEQ ID NO: 16). The centromeric DNA sequence was prepared by PCR using primer 11 (SEQ ID NO: 17), primer 12 (SEQ ID NO: 18), primer 13 (SEQ ID NO: 19), and primer 14 (SEQ ID NO: 20). PARS1 was prepared by PCR using primer 26 (SEQ ID NO: 38) and primer 27 (SEQ ID NO: 39).

A promoter-regulated Zeocin (tradename)-resistance gene (SEQ ID NO: 21), which was used for vector construction, was prepared by PCR using synthetic DNA as a template. A G418-resistance gene (SEQ ID NO: 22), which was used for vector construction, was prepared by PCR using synthetic DNA as a template. A green fluorescent protein gene (SEQ ID NO: 23), which was used for vector construction, was prepared by PCR using synthetic DNA as a template.

PCR was performed by using Prime STAR HS DNA Polymerase (Takara Bio Inc.) etc. under the reaction conditions described in the instructions included. The chromosome DNA was prepared from the Komagataella pastoris strain ATCC76273 using Kaneka Easy DNA Extraction Kit Version 2 (Kaneka Corporation) etc. under the conditions described therein.

### Production Example 2: Construction of Vector for Inactivating the Gene Comprising the Nucleotide Sequence as Shown in SEQ ID NO: 25 (DNL4 Gene)

A nucleic acid fragment in which PstI recognition sequence and BamHI recognition sequence were added to the ends of a DNL4 promoter (SEQ ID NO: 1) was prepared by PCR using primer 1 (SEQ ID NO: 7) and primer 2 (SEQ ID NO: 8). A nucleic acid fragment in which BamHI recognition sequence and KpnI recognition sequence were added to the ends of a DNL4 terminator (SEQ ID NO: 2) was prepared by PCR using primer 3 (SEQ ID NO: 9) and primer 4 (SEQ ID NO: 10). The nucleic acid fragment prepared from the DNL4 promoter was treated with PstI and BamHI, the nucleic acid fragment prepared from the DNL4 terminator was treated with BamHI and KpnI, and the resultants were inserted into a site between PstI and KpnI of pUC19 (Code No. 3219, Takara Bio Inc.). Thus, pUC-Pdn14Tdn14 was constructed.

Subsequently, a nucleic acid fragment in which KpnI recognition sequences were added to the both sides of the promoter-regulated ADE1 gene sequence (SEQ ID NO: 3) was prepared by PCR using primer 5 (SEQ ID NO: 11) and primer 6 (SEQ ID NO: 12), treated with KpnI, and inserted into a KpnI site of pUC-Pdn14Tdn14. Thus, pUC-Pdn14Tdn14ADE1 was constructed.

This vector comprises, as homologous regions, a 1,000-bp promoter region and a 1,007-bp terminator region of the gene (DNL4 gene) comprising the nucleotide sequence as shown in SEQ ID NO: 25 encoding the amino acid sequence (Accession No. CCA39424) as shown in SEQ ID NO: 24, which is DNL4 of the Komagataella pastoris strain ATCC76273, and a promoter-regulated ADE1 gene added thereto. As described in the examples below, this vector is designed to inactivate the gene (DNL4 gene) comprising the nucleotide sequence as shown in SEQ ID NO: 25 by transforming the vector into the host methanol-utilizing yeast strain.

### Production Example 3: Construction of the Autonomously Replicating Vector Expressing the Green Fluorescent Protein

A nucleic acid fragment in which HindIII-XbaI-NotI-AscI-SfiI-PacI-AsiSI-SfiI recognition sequence and EcoRI recognition sequence were added to the ends of the promoter-regulated Zeocin (tradename)-resistance gene (SEQ ID NO: 21) was prepared by PCR using primer 15 (SEQ ID NO: 26) and primer 16 (SEQ ID NO: 27), treated with HindIII and EcoRI, and inserted into a site between HindIII and EcoRI of pUC19. Thus, pUC-Zeo was constructed.

Subsequently, a nucleic acid fragment in which AscI recognition sequence and SpeI recognition sequence were added to the ends of the GAP promoter (SEQ ID NO: 4) was prepared by PCR using primer 7 (SEQ ID NO: 13) and primer 8 (SEQ ID NO: 14). A nucleic acid fragment in which SpeI recognition sequence and XhoI recognition sequence were added to the ends of the green fluorescent protein gene (SEQ ID NO: 23) was prepared by PCR using primer 17 (SEQ ID NO: 28) and primer 18 (SEQ ID NO: 29). A nucleic acid fragment in which XhoI recognition sequence and PacI recognition sequence were added to the ends of GAP1 terminator (SEQ ID NO: 5) was prepared by PCR using primer 9 (SEQ ID NO: 15) and primer 10 (SEQ ID NO: 16). The GAP promoter was treated with AscI and SpeI, the green fluorescent protein gene was treated with SpeI and XhoI, and the GAP1 terminator was treated with XhoI and PacI. The resultants were inserted into a site between AscI and PacI of pUC-Zeo. Thus, pUC-PgapGFPTgap1Zeo was constructed.

Subsequently, a region approximately a half of the centromeric DNA sequence (SEQ ID NO: 6) (i.e., LOR_CC) was prepared by PCR using primer 11 (SEQ ID NO: 17) and primer 12 (SEQ ID NO: 18). A remaining region of an approximately half of the centromeric DNA sequence (i.e., CC_ROR) was prepared by PCR using primer 13 (SEQ ID NO: 19) and primer 14 (SEQ ID NO: 20). LOR_CC was treated with NotI and PstI, CC_ROR was treated with PstI and XbaI, and the resultants were inserted into a site between XbaI and NotI of pUC-PgapGFPTgap1Zeo. Thus, pUC-Cen2PgapGFPTgap1Zeo was constructed.

This vector comprising a centromeric DNA sequence including the autonomously replicating sequence (ARS) of Komagataella pastoris autonomously replicates. In this vector, the green fluorescent protein is regulated by the GAP promoter. A transformant transformed with this vector has resistance to Zeocin (tradename).

PARS1 (SEQ ID NO: 37) was prepared by PCR using primer 26 (SEQ ID NO: 38) and primer 27 (SEQ ID NO: 39), treated with NotI, and inserted into the NotI site of pUC-PgapGFPTgap1Zeo. Thus, pUC-PARS1PgapGFPTgap1Zeo was constructed.

This vector comprising PARS1, which is ARS of Komagataella pastoris, autonomously replicates. In this vector, the green fluorescent protein is regulated by the GAP promoter. A transformant transformed with this vector has resistance to Zeocin (tradename).

### Production Example 4: Construction of the G418-Resistance Gene Vector

A nucleic acid fragment of the G418-resistance gene (SEQ ID NO: 22) was prepared by PCR using primer 19 (SEQ ID NO: 30) and primer 20 (SEQ ID NO: 31). Thus, 0_G418_0 was constructed.

A nucleic acid fragment of the G418-resistance gene (SEQ ID NO: 22) was prepared by PCR using primer 21 (SEQ ID NO: 32) and primer 22 (SEQ ID NO: 33). Thus, 30_G418_30 was constructed. This vector is designed to comprise, at its end, a 30-bp nucleotide sequence exhibiting 100% sequence identity to the GAP promoter and the GAP1 terminator.

A nucleic acid fragment of the G418-resistance gene (SEQ ID NO: 22) was prepared by PCR using primer 23 (SEQ ID NO: 34) and primer 24 (SEQ ID NO: 35). Thus, 60_G418_60 was constructed. This vector is designed to comprise, at its end, a 60-bp nucleotide sequence exhibiting 100% sequence identity to the GAP promoter and the GAP1 terminator.

### Production Example 5: Construction of an Autonomously Replicating Vector Having a Multiple Cloning Site

A nucleic acid fragment in which AOX1 promoter (SEQ ID NO: 41) and GAP1 terminator (SEQ ID NO: 5) were added to the ends of the multiple cloning site (Acc65I-AvrII-EcoRV-MluI-BsrGI) (SEQ ID NO: 40) was prepared by PCR using synthetic DNA as a template, primer 28 (SEQ ID NO: 42), and primer 29 (SEQ ID NO: 43). The nucleic acid fragment was treated with AscI and PacI and inserted into a site between AscI and PacI of pUC-Cen2PgapGFPTgap1Zeo constructed in Production Example 3. Thus, pUC-Cen2Paox1MCSTgap1Zeo was constructed. This vector comprising a centromeric DNA sequence including the autonomously replicating sequence (ARS) of Komagataella pastoris autonomously replicate. This vector further comprises a multiple cloning site (Acc65I-AvrII-EcoRV-MluI-BsrGI) downstream of the AOX1 promoter and the GAP1 terminator downstream of the multiple cloning site. A transformant transformed with this vector has resistance to Zeocin (tradename).

### Production Example 6: Construction of Vectors

A nucleic acid fragment comprising positions 855449 to 856477 of Chromosome No. 6 (AECK01000006) was prepared by PCR using, as a template, a mixture of chromosome DNA from the Ogataea angusta strain NCYC495 (the nucleotide sequences thereof are described in EMBL (The European Molecular Biology Laboratory) Accession Nos. AECK01000001 to AECK01000007), primer 30 (SEQ ID NO: 44), and primer 31 (SEQ ID NO: 45). Thus, 1,106-bp Fragment 1 (SEQ ID NO: 46) was constructed.

Also, a nucleic acid fragment comprising positions 856357 to 858859 of Chromosome No. 6 (AECK01000006) was prepared by PCR using primer 32 (SEQ ID NO: 47) and primer 33 (SEQ ID NO: 48). Thus, 2,503-bp Fragment 2 (SEQ ID NO: 49) was constructed.

Also, a nucleic acid fragment comprising positions 858744 to 861310 of Chromosome No. 6 (AECK01000006) was prepared by PCR using primer 34 (SEQ ID NO: 50) and primer 35 (SEQ ID NO: 51). Thus, 2,567-bp Fragment 3 (SEQ ID NO: 52) was constructed.

Also, a nucleic acid fragment comprising positions 861181 to 863668 of Chromosome No. 6 (AECK01000006) was prepared by PCR using primer 36 (SEQ ID NO: 53) and primer 37 (SEQ ID NO: 54). Thus, 2.488-bp Fragment 4 (SEQ ID NO: 55) was constructed.

Also, a nucleic acid fragment comprising positions 863538 to 866113 of Chromosome No. 6 (AECK01000006) was prepared by PCR using primer 38 (SEQ ID NO: 56) and primer 39 (SEQ ID NO: 57). Thus, 2.624-bp Fragment 5 (SEQ ID NO: 58) was constructed.

A nucleic acid fragment comprising a nucleotide sequence of positions 1 to 195 of the green fluorescent protein gene to which the GAP promoter (SEQ ID NO: 4) was ligated was prepared by PCR using synthetic DNA as a template, primer 40 (SEQ ID NO: 59), and primer 41 (SEQ ID NO: 60). Thus, 735-bp PgapGFP1 (SEQ ID NO: 61) was constructed.

A nucleic acid fragment comprising a nucleotide sequence of nucleotides 101 to 720 of the green fluorescent protein gene was prepared by PCR using synthetic DNA as a template, primer 42 (SEQ ID NO: 62), and primer 43 (SEQ ID NO: 63). Thus, 696-bp GFP2 (SEQ ID NO: 64) was constructed.

Fragment 1 is designed to comprise, at the upper end, a 77-bp nucleotide sequence exhibiting 100% sequence identity to the AOX1 promoter and the multiple cloning site and, at the lower end, a 121-bp nucleotide sequence exhibiting 100% sequence identity to Fragment 2.

Fragment 2 is designed to comprise, at the upper end, a 121-bp nucleotide sequence exhibiting 100% sequence identity to Fragment 1 and, at the lower end, a 116-bp nucleotide sequence exhibiting 100% sequence identity to Fragment 3.

Fragment 3 is designed to comprise, at the upper end, a 116-bp nucleotide sequence exhibiting 100% sequence identity to Fragment 2 and, at the lower end, a 130-bp nucleotide sequence exhibiting 100% sequence identity to Fragment 4.

Fragment 4 is designed to comprise, at the upper end, a 130-bp nucleotide sequence exhibiting 100% sequence identity to Fragment 3 and, at the lower end, a 131-bp nucleotide sequence exhibiting 100% sequence identity to Fragment 5.

Fragment 5 is designed to comprise, at the upper end, a 131-bp nucleotide sequence exhibiting 100% sequence identity to Fragment 4 and, at the lower end, a 95-bp nucleotide sequence exhibiting 100% sequence identity to PgapGFP1.

PgapGFP1 is designed to comprise, at the upper end, a 95-bp nucleotide sequence exhibiting 100% sequence identity to Fragment 5 and, at the lower end, a 95-bp nucleotide sequence exhibiting 100% sequence identity to GFP2.

GFP2 is designed to comprise, at the upper end, a 95-bp nucleotide sequence exhibiting 100% sequence identity to PgapGFP1 and, at the lower end, a 76-bp nucleotide sequence exhibiting 100% sequence identity to the multiple cloning site and the GAP1 terminator.

TABLE 1

| | | Primer sequence | Primer sequence | | | |
|---|---|---|---|---|---|---|
| | | (Fw) used for | (Re) used for | | | |
| | | amplification | amplification | Position of | | |
| | Nucleotide | of nucleic | of nucleic | Chromosome 6 | | |
| Vector | sequence | acid fragment | acid fragment | (AECK01000006) | Length | |
| Fragment 1 | SEQ ID | Primer 3 | Primer 31 | 855449-856477 | 1,106 | bp |
| | NO: 46 | (SEQ ID NO: 44) | (SEQ ID NO: 45) | | | |
| Fragment 2 | SEQ ID | Primer 32 | Primer 33 | 856357-858859 | 2,503 | bp |
| | NO: 49 | (SEQ ID NO: 47) | (SEQ ID NO: 48) | | | |
| Fragment 3 | SEQ ID | Primer 34 | Primer 35 | 858744-861310 | 2,567 | bp |
| | NO: 52 | (SEQ ID NO: 50) | (SEQ ID NO: 51) | | | |
| Fragment 4 | SEQ ID | Primer 36 | Primer 37 | 861181-863668 | 2,488 | bp |
| | NO: 55 | (SEQ ID NO: 53) | (SEQ ID NO: 54) | | | |
| Fragment 5 | SEQ ID | Primer 38 | Primer 39 | 863538-866113 | 2,624 | bp |
| | NO: 58 | (SEQ ID NO: 56) | (SEQ ID NO: 57) | | | |
| PgapGFP1 | SEQ ID | Primer 40 | Primer 41 | — | 735 | bp |
| | NO: 61 | (SEQ ID NO: 59) | (SEQ ID NO: 60) | | | |
| GFP2 | SEQ ID | Primer 42 | Primer 43 | — | 696 | bp |
| | NO: 64 | (SEQ ID NO: 62) | (SEQ ID NO: 63) | | | |

### Example 1: Generation of Transformed Yeast in which the Gene Comprising the Nucleotide Sequence as Shown in SEQ ID NO: 25 (DNL4 Gene) is Inactivated

With the use of the vector for inactivating the gene comprising the nucleotide sequence as shown in SEQ ID NO: 25 (DNL4 gene) constructed in Production Example 2 (pUC-Pdn14Tdn14ADE1), a host methanol-utilizing yeast strain was transformed in the manner described below.

An adenine auxotrophic strain derived from the Komagataella pastoris strain ATCC76273 was inoculated in 3 ml of YPD medium (1% yeast extract bacto (Becton, Dickinson and Company), 2% hipolypeptone (Nihon Pharmaceutical Co., Ltd.), and 2% glucose) and shake-cultured overnight at 30° C. to obtain a preculture suspension. The preculture suspension thus obtained (500 μl) was inoculated in 50 ml of YPD medium and shake-cultured up to an OD600 of 1 to 1.5. Thereafter, the yeast culture was harvested (3000×g, 10 minutes, 20° C.) and resuspended in 10 ml of 50 mM potassium phosphate buffer (pH 7.5) supplemented with 250 μl of 1 M DTT (final concentration: 25 mM).

After the suspension was subjected to incubation for 15 minutes at 30° C., the yeast culture was harvested (3000×g, 10 minutes, 20° C.) and washed with 50 ml of STM buffer precooled in ice (270 mM sucrose, 10 mM Tris-HCl, 1 mM magnesium chloride, pH 7.5). The culture was harvested from the washing solution (3000×g, 10 minutes, 4° C.) and washed again with 25 ml of STM buffer, followed by harvesting (3000×g, 10 minutes, 4° C.). In the end, the obtained yeast culture was suspended in 250 μl of the ice-cold STM buffer and the resulting suspension was designated as a competent cell suspension.

With the use of the vector for inactivating the gene comprising the nucleotide sequence as shown in SEQ ID NO: 25 (DNL4 gene) constructed in Production Example 2 (pUC-Pdn14Tdn14ADE1), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Pdn14Tdn14ADE1 was obtained from the cultured cells using FastGene Plasmid Mini Kit (NIPPON Genetics Co., Ltd.). This plasmid was linearized via BglII treatment targeting a BglII recognition sequence within the DNL4 terminator.

The competent cell suspension obtained above (60 μl) was mixed with 3 μl of a solution of the linearized pUC-Pdn14Tdn14ADE1, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.) and suspended in 1 ml of YNB medium (0.67% Yeast Nitrogen Base Without Amino Acids (Becton, Dickinson and Company). Thereafter, the yeast culture was harvested again (3000×g, 5 minutes, 20° C.), resuspended in an adequate amount of YNB medium, and then spread on a selective YNB agar plate (0.67% Yeast Nitrogen Base Without Amino Acids (Becton, Dickinson and Company), 1.5% agarose, and 2% glucose). A strain that grew in static culture for 3 days at 30° C. was selected.

With the use of the selected strain, homologous recombination was performed to delete the gene comprising the nucleotide sequence as shown in SEQ ID NO: 25 (DNL4 gene) and the promoter-regulated ADE1 gene. Thus, the Δdn14Δade1 strain was obtained.

Subsequently, the ADE1 gene was introduced into the HIS4 gene region of the Δdn14Δade1 strain to obtain the Δdn14Δhis4 strain. This strain is a histidine auxotrophic transformed yeast strain in which the gene comprising the nucleotide sequence as shown in SEQ ID NO: 25 (DNL4 gene) has been inactivated.

### Comparative Example 1: Generation of Transformed Yeast, Calculation of Transformation Efficiency, and Confirmation of Self-Circularization

With the use of the autonomously replicating vector expressing the green fluorescent protein constructed in Production Example 3 (pUC-Cen2PgapGFPTgap1Zeo), the wild-type strain of the Komagataella pastoris strain ATCC76273 was transformed in the manner described below.

The wild-type strain was inoculated in 3 ml of YPD medium (1% yeast extract bacto (Becton, Dickinson and Company), 2% hipolypeptone (Nihon Pharmaceutical Co., Ltd.), and 2% glucose) and shake-cultured overnight at 30° C. to obtain a preculture suspension. The preculture suspension thus obtained (500 μl) was inoculated in 50 ml of YPD medium and shake-cultured up to an OD600 of 1 to 1.5. Thereafter, the yeast culture was harvested (3000×g, 10 minutes, 20° C.) and resuspended in 10 ml of 50 mM potassium phosphate buffer (pH 7.5) supplemented with 250 μl of 1M DTT (final concentration: 25 mM).

After the suspension was subjected to incubation for 15 minutes at 30° C., the yeast culture was harvested (3000×g, 10 minutes, 20° C.) and washed with 50 ml of STM buffer precooled in ice (270 mM sucrose, 10 mM Tris-HCl, 1 mM magnesium chloride, pH 7.5). The culture was harvested from the washing solution (3000×g, 10 minutes, 4° C.) and washed again with 25 ml of STM buffer, followed by harvesting (3000×g, 10 minutes, 4° C.). In the end, the obtained yeast culture was suspended in 250 μl of the ice-cold STM buffer and the resulting suspension was designated as a competent cell suspension.

With the use of the autonomously replicating vector expressing the green fluorescent protein constructed in Production Example 3 (pUC-Cen2PgapGFPTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2PgapGFPTgap1Zeo was obtained from the cultured cells using FastGene Plasmid Mini Kit (NIPPON Genetics Co., Ltd.). This plasmid was linearized via BsrGI treatment targeting a BsrGI recognition sequence within the green fluorescent protein gene.

The competent cell suspension obtained above (60 μl) was mixed with 3 μl of a solution of the linearized pUC-Cen2PgapGFPTgap1Zeo, or the competent cell suspension obtained above (60 μl) was mixed with 3 μl of a solution of the pUC-Cen2PgapGFPTgap1Zeo that was not subjected to BsrGI treatment, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution and then spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector; cfu/μg) (Table 2).

TABLE 2

| | | | Transformation |
|---|---|---|---|
| | | | efficiency |
| Condition | Host | Vector | (cfu/μg) |
| 1 | Wild-type | pUC-Cen2PgapGFPTgap1Zeo | 8.1 × 10³ |
| | | (treated with BsrGI) | |
| 2 | Wild-type | pUC-Cen2PgapGFPTgap1Zeo | 7.1 × 10³ |
| | | (not treated with BsrGI) | |

As a result, transformation efficiency of Condition 1 attained with the use of pUC-Cen2PgapGFPTgap1Zeo linearized via BsrGI treatment was found to be equivalent to transformation efficiency of Condition 2 attained with the use of pUC-Cen2PgapGFPTgap1Zeo that was not subjected to BsrGI treatment. This indicates that self-circularization of the linearized pUC-Cen2PgapGFPTgap1Zeo has occurred because the wild-type strain has non-homologous end joining activity.

The 10 transformed yeast strains obtained under Condition 1 were inoculated on a selective YPD Zeocin (tradename) agar plate and subjected to static culture at 30° C. for 1 day. A plasmid solution was obtained from the grown strains using Easy Yeast Plasmid Isolation Kit (Clontech Laboratories, Inc.) and then introduced into E. coli HST08 competent cells (Takara Bio Inc.). The E. coli transformants were cultured to amplify the plasmid, and then the plasmids were subjected to electrophoresis, sequence analysis, and other analytical techniques. As a result, it was found that self-circularization of pUC-Cen2PgapGFPTgap1Zeo linearized via BsrGI treatment had occurred in all samples.

### Example 2: Generation of Transformed Yeast and Calculation of Transformation Efficiency

With the use of the autonomously replicating vector expressing the green fluorescent protein constructed in Production Example 3 (pUC-Cen2PgapGFPTgap1Zeo), the histidine auxotrophic transformed yeast strain Δdn14Δhis4 constructed in Example 1 in which the gene comprising the nucleotide sequence as shown in SEQ ID NO: 25 (DNL4 gene) had been inactivated was transformed.

The Δdn14Δhis4 strain was inoculated in 3 ml of YPD medium (1% yeast extract bacto (Becton, Dickinson and Company), 2% hipolypeptone (Nihon Pharmaceutical Co., Ltd.), and 2% glucose) and shake-cultured overnight at 30° C. to obtain a preculture suspension. The preculture suspension thus obtained (500 μl) was inoculated in 50 ml of YPD medium and shake-cultured up to an OD600 of 1 to 1.5. Thereafter, the yeast culture was harvested (3000×g, 10 minutes, 20° C.) and resuspended in 10 ml of 50 mM potassium phosphate buffer (pH 7.5) supplemented with 250 μl of 1M DTI (final concentration: 25 mM).

After the suspension was subjected to incubation for 15 minutes at 30° C., the yeast culture was harvested (3000×g, 10 minutes, 20° C.) and washed with 50 ml of STM buffer precooled in ice (270 mM sucrose, 10 mM Tris-HCl, 1 mM magnesium chloride, pH 7.5). The culture was harvested from the washing solution (3000×g, 10 minutes, 4° C.) and washed again with 25 ml of STM buffer, followed by harvesting (3000×g, 10 minutes, 4° C.). In the end, the obtained yeast culture was suspended in 250 μl of the ice-cold STM buffer and the resulting suspension was designated as a competent cell suspension.

With the use of the autonomously replicating vector expressing the green fluorescent protein constructed in Production Example 3 (pUC-Cen2PgapGFPTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2PgapGFPTgap1Zeo was obtained from the cultured cells using FastGene Plasmid Mini Kit (NIPPON Genetics Co., Ltd.). This plasmid was linearized via BsrGI treatment targeting a BsrGI recognition sequence within the green fluorescent protein gene.

The competent cell suspension obtained above (60 μl) was mixed with 3 μl of a solution of the linearized pUC-Cen2PgapGFPTgap1Zeo, or the competent cell suspension obtained above (60 μl) was mixed with 3 μl of a solution of the pUC-Cen2PgapGFPTgap1Zeo that was not subjected to BsrGI treatment, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200 S, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution, and the suspension was spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector; cfu/μg) (Table 3).

TABLE 3

| | | | Transformation |
|---|---|---|---|
| | | | efficiency |
| Condition | Host | Vector | (cfu/μg) |
| 3 | Δdnl4Δhis4 | pUC-Cen2PgapGFPTgap1Zeo | 0.0 |
| | | (treated with BsrGI) | |
| 4 | Δdnl4Δhis4 | pUC-Cen2PgapGFPTgap1Zeo | 2.4 × 10³ |
| | | (not treated with BsrGI) | |

As a result, transformation efficiency of Condition attained 3 with the use of pUC-Cen2PgapGFPTgap1Zeo linearized via BsrGI treatment was found to have decreased to a significant extent, compared with transformation efficiency of Condition 4 attained with the use of pUC-Cen2PgapGFPTgap1Zeo that was not subjected to BsrGI treatment, and no colonies were generated. This indicates that the Δdn14Δhis4 strain has lost non-homologous end joining activity and self-circularization of the linearized pUC-Cen2PgapGFPTgap1Zeo is completely suppressed upon inactivation of the gene comprising the nucleotide sequence as shown in SEQ ID NO: 25 (DNL4 gene).

### Comparative Example 2: Generation of Transformed Yeast, Calculation of Transformation Efficiency, and Calculation of Assembly Efficiency

In order to assemble vectors, the autonomously replicating vector expressing the green fluorescent protein constructed in Production Example 3 (pUC-Cen2PgapGFPTgap1Zeo) and the G418-resistance gene vector constructed in Production Example 4 (60_G418_60) were used to transform the wild-type strain of the Komagataella pastoris strain ATCC76273 in the manner described below.

The wild-type strain was inoculated in 3 ml of YPD medium (1% yeast extract bacto (Becton, Dickinson and Company), 2% hipolypeptone (Nihon Pharmaceutical Co., Ltd.), and 2% glucose) and shake-cultured overnight at 30° C. to obtain a preculture suspension. The preculture suspension thus obtained (500 μl) was inoculated in 50 ml of YPD medium and shake-cultured up to an OD600 of 1 to 1.5. Thereafter, the yeast culture was harvested (3000×g, 10 minutes, 20° C.) and resuspended in 10 ml of 50 mM potassium phosphate buffer (pH 7.5) supplemented with 250 μl of 1M DTT (final concentration: 25 mM).

After the suspension was subjected to incubation for 15 minutes at 30° C., the yeast culture was harvested (3000×g, 10 minutes, 20° C.) and washed with 50 ml of STM buffer precooled in ice (270 mM sucrose, 10 mM Tris-HCl, 1 mM magnesium chloride, pH 7.5). The culture was harvested from the washing solution (3000×g, 10 minutes, 4° C.) and washed again with 25 ml of STM buffer, followed by harvesting (3000×g, 10 minutes, 4° C.). In the end, the obtained yeast culture was suspended in 250 μl of the ice-cold STM buffer and the resulting suspension was designated as a competent cell suspension.

With the use of the autonomously replicating vector expressing the green fluorescent protein constructed in Production Example 3 (pUC-Cen2PgapGFPTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2PgapGFPTgap1Zeo was obtained from the cultured cells using FastGene Plasmid Mini Kit (NIPPON Genetics Co., Ltd.). This plasmid was linearized via BsrGI treatment targeting a BsrGI recognition sequence within the green fluorescent protein gene.

The competent cell suspension obtained above (60 μl), a solution of the linearized pUC-Cen2PgapGFPTgap1Zeo (0.015 pmol), and the G418-resistance gene vector 60_G418_60 (0.75 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 μF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution and spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector, cfu/μg) (Table 4, Condition 5).

The 20 transformed yeast strains obtained under Condition 5 were inoculated on a selective YPD Zeocin (tradename) agar plate and subjected to static culture at 30° C. for 1 day. In order to confirm that vectors had been assembled, chromosome DNA and a plasmid solution were obtained from the grown strains using Kaneka Easy DNA Extraction Kit Version 2 (Kaneka Corporation) and subjected to PCR with the use thereof as a template, primer 19 (SEQ ID NO: 30) as a forward primer for the G418-resistance gene, and primer 25 (SEQ ID NO: 36) as a reverse primer at a position 156 bp away from the origin of the GAP1 terminator. The PCR product was electrophoresed, and assembly efficiency was calculated based on the number of samples in which a band of the size of interest (951 bp) had developed (Table 4, Condition 5).

### Example 3: Generation of Transformed Yeast, Calculation of Transformation Efficiency, and Calculation of Assembly Efficiency

In order to assemble vectors, the autonomously replicating vectors expressing the green fluorescent protein constructed in Production Example 3 (pUC-Cen2PgapGFPTgap1Zeo and pUC-PARS1PgapGFPTgap1Zeo) and the G418-resistance gene vectors constructed in Production Example 4 (0_G418_0, 30_G418_30, and 60_G418_60) were used to transform the histidine auxotrophic transformed yeast strain Δdn14Δhis4 constructed in Example 1 in which the gene comprising the nucleotide sequence as shown in SEQ ID NO: 25 (DNL4 gene) had been inactivated in the manner described below.

The Δdn14Δhis4 strain was inoculated in 3 ml of YPD medium (1% yeast extract bacto (Becton. Dickinson and Company), 2% hipolypeptone (Nihon Pharmaceutical Co., Ltd.), and 2% glucose) and shake-cultured overnight at 30° C. to obtain a preculture suspension. The preculture suspension thus obtained (500 μl) was inoculated in 50 ml of YPD medium and shake-cultured up to an OD600 of 1 to 1.5. Thereafter, the yeast culture was harvested (3000×g, 10 minutes, 20° C.) and resuspended in 10 ml of 50 mM potassium phosphate buffer (pH 7.5) supplemented with 250 μl of 1M DTT (final concentration: 25 mM).

After the suspension was subjected to incubation for 15 minutes at 30° C., the yeast culture was harvested (3000×g, 10 minutes, 20° C.) and washed with 50 ml of STM buffer precooled in ice (270 mM sucrose, 10 mM Tris-HCl, 1 mM magnesium chloride, pH 7.5). The culture was harvested from the washing solution (3000×g, 10 minutes, 4° C.) and washed again with 25 ml of STM buffer, followed by harvesting (3000×g, 10 minutes, 4° C.). In the end, the obtained yeast culture was suspended in 250 μl of the ice-cold STM buffer and the resulting suspension was designated as a competent cell suspension.

With the use of the autonomously replicating vector expressing the green fluorescent protein constructed in Production Example 3 (pUC-Cen2PgapGFPTgap1Zeo or pUC-PARS1PgapGFPTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2PgapGFPTgap1Zeo or pUC-PARS1PgapGFPTgap1Zeo was obtained from the cultured cells using FastGene Plasmid Mini Kit (NIPPON Genetics Co., Ltd.). This plasmid was linearized via BsrGI treatment targeting a BsrGI recognition sequence within the green fluorescent protein gene.

The competent cell suspension obtained above (60 μl), a solution of linearized pUC-Cen2PgapGFPTgap1Zeo (0.015 pmol) or a solution of linearized pUC-PARS1PgapGFPTgap1Zeo (0.015 pmol), and the G418-resistance gene vector 0_G418_0, 30_G418_30, or 60_G418_60 (0.15 pmol or 0.75 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 μF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution and spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector; cfu/μg) (Table 4, Conditions 6 to 12).

The 10 transformed yeast strains obtained under Conditions 8 and 9, the 20 transformed yeast strains obtained under Conditions 10 and 11, and the 10 transformed yeast strains obtained under Condition 12 were inoculated on a selective YPD Zeocin (tradename) agar plate and subjected to static culture at 30° C. for 1 day. In order to confirm that vectors had been assembled, chromosome DNA and a plasmid solution were obtained from the grown strains using Kaneka Easy DNA Extraction Kit Version 2 (Kaneka Corporation) and subjected to PCR with the use thereof as a template, primer 19 (SEQ ID NO: 30) as a forward primer for the G418-resistance gene, and primer 25 (SEQ ID NO: 36) as a reverse primer at a position 156 bp away from the origin of the GAP1 terminator. The PCR product was electrophoresed, and assembly efficiency was calculated based on the number of samples in which a band of the size of interest (951 bp) had developed (Table 4, Conditions 8 to 12).

TABLE 4

| | | | | Molar ratio | Transformation | Assembly |
|---|---|---|---|---|---|---|
| | | | | (Vector | efficiency | efficiency |
| Condition | Host | Vector (1) | Vector (2) | (1):Vector (2)) | (cfu/μg) | (%) |
| 5 | Wild-type | pUC-Cen2PgapGFPTgap1Zeo | 60_G418_60 | 1:50 | 1.1 × 10⁴ | 20 |
| | | (treated with BsrGI) | | | | |
| 6 | Δdnl4Δhis4 | pUC-Cen2PgapGFPTgap1Zeo | 0_G418_0 | 1:10 | 0 | — |
| | | (treated with BsrGI) | | | | |
| 7 | Δdnl4Δhis4 | pUC-Cen2PgapGFPTgap1Zeo | 0_G418_0 | 1:50 | 0 | — |
| | | (treated with BsrGI) | | | | |
| 8 | Δdnl4Δhis4 | pUC-Cen2PgapGFPTgap1Zeo | 30_G418_30 | 1:10 | 50 | 80 |
| | | (treated with BsrGI) | | | | |
| 9 | Δdnl4Δhis4 | pUC-Cen2PgapGFPTgap1Zeo | 30_G418_30 | 1:50 | 50 | 100 |
| | | (treated with BsrGI) | | | | |
| 10 | Δdnl4Δhis4 | pUC-Cen2PgapGFPTgap1Zeo | 60_G418_60 | 1:10 | 2.4 × 10² | 95 |
| | | (treated with BsrGI) | | | | |
| 11 | Δdnl4Δhis4 | pUC-Cen2PgapGFPTgap1Zeo | 60_G418_60 | 1:50 | 9.2 × 10³ | 100 |
| | | (treated with BsrGI) | | | | |
| 12 | Δdnl4Δhis4 | pUC-PARS1PgapGFPTgap1Zeo | 60_G418_60 | 1:50 | 9.1 × 10³ | 100 |
| | | (treated with BsrGI) | | | | |

As a result, the assembly efficiency of Condition 5 attained with the use of pUC-Cen2PgapGFPTgap1Zeo linearized via BsrGI treatment and the G418-resistance gene vector 60_G418_60 designed to comprise a 60-bp nucleotide sequence exhibiting 100% sequence identity to the GAP promoter and the GAP1 terminator was found to be as low as 20%. This indicates that 60_G418_60 is not efficiently assembled in the wild-type strain due to self-circularization of linearized pUC-Cen2PgapGFPTgap1Zeo because the wild-type strain has non-homologous end joining activity.

Condition 6 and Condition 7 are each prepared via transformation into the host Δdn14Δhis4 strain with the use of pUC-Cen2PgapGFPTgap1Zeo linearized via BsrGI treatment and the G418-resistance gene vector 0_G418_0 that is not designed to comprise a nucleotide sequence exhibiting sequence identity to the GAP promoter and the GAP1 terminator. As with the results shown in Table 3 in Example 3, self-circularization of linearized pUC-Cen2PgapGFPTgap1Zeo was suppressed.

Meanwhile, the assembly efficiency of Conditions 8 to 11 attained with the use of linearized pUC-Cen2PgapGFPTgap1Zeo and the G418-resistance gene vectors 30_G418_30 and 60_G418_60 was high. This indicates that the Δdn14Δhis4 strain has lost non-homologous end joining activity upon inactivation of the gene comprising the nucleotide sequence as shown in SEQ ID NO: 25 (DNL4 gene), self-circularization of linearized pUC-Cen2PgapGFPTgap1Zeo has been completely suppressed, and the vectors are efficiently assembled in the yeast strains.

Assembly efficiency of Condition 12 attained with the use of linearized pUC-PARS1PgapGFPTgap1Zeo and the G418-resistance gene vector 60_G418_60 was high. This indicates that vectors are assembled with each other efficiently in yeast regardless of a difference in ARS because the Δdn14Δhis4 strain has lost non-homologous end joining activity upon inactivation of the gene comprising the nucleotide sequence as shown in SEQ ID NO: 25 (DNL4 gene) and self-circularization of linearized pUC-PARS1PgapGFPTgap1Zeo has been completely suppressed.

A plasmid solution was obtained from the strain in which vector assembly was confirmed via PCR using primer 19 (SEQ ID NO: 30) and primer 25 (SEQ ID NO: 36) using Easy Yeast Plasmid Isolation Kit (Clontech Laboratories, Inc.) and introduced into E. coli HST08 competent cells (Takara Bio Inc.). The E. coli transformants were cultured to amplify plasmids, and the plasmids were subjected to sequence analysis. As a result, it was found that the green fluorescent protein gene of pUC-Cen2PgapGFPTgap1Zeo linearized via BsrGI treatment had been substituted with the G418-resistance gene in all samples.

### Comparative Example 3: Generation of Transformed Yeast, Calculation of Transformation Efficiency, and Calculation of Assembly Efficiency

In order to assemble vectors, the wild-type strain of the Komagataella pastoris strain ATCC76273 was transformed in the same manner as in Comparative Example 2 with the use of the autonomously replicating vector having a multiple cloning site constructed in Production Example 5 (pUC-Cen2Paox1MCSTgap1Zeo), Fragment 1, Fragment 2, Fragment 3, Fragment 4, Fragment 5, PgapGFP1, and GFP2 constructed in Production Example 6.

With the use of the autonomously replicating vector having a multiple cloning site constructed in Production Example 5 (pUC-Cen2Paox1MCSTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2Paox1MCSTgap1Zeo was obtained from the cultured cells using FastGene Plasmid Mini Kit (NIPPON Genetics Co., Ltd.). This plasmid was linearized via MluI treatment digesting a MluI recognition sequence within the multiple cloning site.

The competent cell suspension obtained in Comparative Example 2 (60 μl), a solution of the linearized pUC-Cen2Paox1MCSTgap1Zeo (0.015 pmol), a solution of Fragment 1 constructed in Production Example 6 (0.15 pmol), a solution of Fragment 2 constructed in Production Example 6 (0.15 pmol), a solution of Fragment 3 constructed in Production Example 6 (0.15 pmol), a solution of Fragment 4 constructed in Production Example 6 (0.15 pmol), a solution of Fragment 5 constructed in Production Example 6 (0.15 pmol), a solution of PgapGFP1 constructed in Production Example 6 (0.15 pmol), and a solution of GFP2 constructed in Production Example 6 (0.15 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution and spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector; cfu/μg) (Table 5, Condition 13).

The 10 transformed yeast strains obtained under Condition 13 were inoculated on a selective YPD Zeocin (tradename) agar plate and subjected to static culture at 30° C. for 1 day. In order to confirm that vectors had been assembled, chromosome DNA and a plasmid solution were obtained from the grown strains using Kaneka Easy DNA Extraction Kit Version 2 (Kaneka Corporation) and subjected to PCR with the use thereof as a template, primer 44 (SEQ ID NO: 65) as a forward primer at a position 920 bp away from the origin of the AOX1 promoter, and primer 25 (SEQ ID NO: 36) as a reverse primer at a position 156 bp away from the origin of the GAP1 terminator. The PCR product was electrophoresed, and assembly efficiency was calculated based on the number of samples in which a band of the size of interest (about 12 kbp) had developed (Table 5, Condition 13).

The fluorescence-emitting strains were selected from the 1,123 transformed yeast strains obtained under Condition 13, the number thereof was counted, and the proportion thereof was determined (Table 5, Condition 13).

### Example 4: Generation of Transformed Yeast, Calculation of Transformation Efficiency, and Calculation of Assembly Efficiency

In order to assemble vectors, the autonomously replicating vector having a multiple cloning site constructed in Production Example 5 (pUC-Cen2Paox1MCSTgap1Zeo), Fragment 1, Fragment 2, Fragment 3, Fragment 4, Fragment 5, PgapGFP1, and GFP2 constructed in Production Example 6 were used to transform the histidine auxotrophic transformed yeast strain Δdn14Δhis4 constructed in Example 1 in which the gene comprising the nucleotide sequence as shown in SEQ ID NO: 25 (DNL4 gene) had been inactivated in the same manner as in Example 3.

With the use of the autonomously replicating vector having a multiple cloning site constructed in Production Example 5 (pUC-Cen2Paox1MCSTgap1Zeo), E. coli was transformed, the transformant was cultured in 5 ml of ampicillin-containing 2YT medium (1.6% tryptone bacto (Becton, Dickinson and Company), 1% yeast extract bacto (Becton, Dickinson and Company), 0.5% sodium chloride, and 0.01% ampicillin sodium (Wako Pure Chemical Corporation)), and pUC-Cen2Paox1MCSTgap1Zeo was obtained from the cultured cells using FastGene Plasmid Mini Kit (NIPPON Genetics Co., Ltd.). This plasmid was linearized via MulI treatment targeting a MulI recognition sequence within the multiple cloning site.

The competent cell suspension obtained in Example 3 (60 μl), a solution of the linearized pUC-Cen2Paox1MCSTgap1Zeo (0.015 pmol), a solution of Fragment 1 constructed in Production Example 6 (0.15 pmol), a solution of Fragment 2 constructed in Production Example 6 (0.15 pmol), a solution of Fragment 3 constructed in Production Example 6 (0.15 pmol), a solution of Fragment 4 constructed in Production Example 6 (0.15 pmol), a solution of Fragment 5 constructed in Production Example 6 (0.15 pmol), a solution of PgapGFP1 constructed in Production Example 6 (0.15 pmol), and a solution of GFP2 constructed in Production Example 6 (0.15 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 μF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and 950 μl of the supernatant was discarded. The yeast culture was resuspended in a remaining solution and spread on a selective YPD Zeocin (tradename) agar plate (1% yeast extract (Becton, Dickinson and Company), 2% hipolypeptone (Nihon Pharmaceutical Co., Ltd.), 2% glucose, 1.5% agarose, and 0.01% Zeocin (tradename) (Thermo Fisher Scientific, Inc.)). A strain that grew in static culture for 3 days at 30° C. was selected, and transformation efficiency was calculated (the number of colonies per μg of vector; cfu/μg) (Table 5, Condition 14).

The 10 transformed yeast strains obtained under Condition 14 were inoculated on a selective YPD Zeocin (tradename) agar plate and subjected to static culture at 30° C. for 1 day. In order to confirm that vectors had been assembled, chromosome DNA and a plasmid solution were obtained from the grown strains using Kaneka Easy DNA Extraction Kit Version 2 (Kaneka Corporation) and subjected to PCR with the use thereof as a template, primer 44 (SEQ ID NO: 65) as a forward primer at a position 920 bp away from the origin of the AOX1 promoter, and primer 25 (SEQ ID NO: 36) as a reverse primer at a position 156 bp away from the origin of the GAP1 terminator. The PCR product was electrophoresed, and assembly efficiency was calculated based on the number of samples in which a band of the size of interest (about 12 kbp) had developed (Table 5, Condition 14).

The fluorescence-emitting strains were selected from the 561 transformed yeast strains obtained under Condition 14, the number thereof was counted, and the proportion thereof was determined (Table 5, Condition 14).

TABLE 5

| | | | | | | | Proportion of |
|---|---|---|---|---|---|---|---|
| | | | | Motar ratio | Transformation | Assembly | fluorescence- |
| | | | | (Vector | efficiency | efficiency | emitting |
| Condition | Host | Vector (1) | Vector (2) | (1):Vector (2)) | (cfu/μg) | (%) | strains (%) |
| 13 | Wild-type | pUC-Cen2Paox1MCSTgap1Zeo | Fragments 1 to 5, | 1:10 | 9.7 × 10³ | 20 | 13 |
| | | (treated with Mlul) | PgapGFP1, GFP2 | | | | |
| 14 | Δdnl4Δhis4 | pUC-Cen2Paox1MCSTgap1Zeo | Fragments 1 to 5, | 1:10 | 4.8 × 10³ | 100 | 95 |
| | | (treated with Mlul) | PgapGFP1, GFP2 | | | | |

As a result, the assembly efficiency of Condition 13 attained with the use of pUC-Cen2Paox1MCSTgap1Zeo linearized via MluI treatment, Fragment 1, Fragment 2, Fragment 3, Fragment 4, Fragment 5, PgapGFP1, and GFP2 were found to be as low as 20%, and the proportion of fluorescence-emitting strains was found to be as low as 13%. This indicates that vectors are not efficiently assembled in yeast due to, for example, self-circularization of linearized pUC-Cen2Paox1MCSTgap1Zeo that had occurred because of non-homologous end joining activity of the wild-type strain.

Meanwhile, the assembly efficiency of Condition 14 attained with the use of pUC-Cen2Paox1MCSTgap1Zeo linearized via MluI treatment, Fragment 1, Fragment 2, Fragment 3, Fragment 4, Fragment 5, PgapGFP1, and GFP2 was as high as 100%, and the proportion of fluorescence-emitting strains was as high as 95%. This indicates that vectors are assembled with each other efficiently in yeast because the Δdn14Δhis4 strain has lost non-homologous end joining activity upon inactivation of the gene comprising the nucleotide sequence as shown in SEQ ID NO: 25 (DNL4 gene) and self-circularization of linearized pUC-Cen2Paox1MCSTgap1Zeo has been completely suppressed.

A plasmid solution was obtained from the strain in which vector assembly was confirmed via PCR using primer 44 (SEQ ID NO: 65) and primer 25 (SEQ ID NO: 36) using Easy Yeast Plasmid Isolation Kit (Clontech Laboratories. Inc.) and introduced into E. coli HST08 competent cells (Takara Bio Inc.). The E. coli transformants were cultured to amplify plasmids, and the plasmids were subjected to sequence analysis. As a result, it was found that 7 nucleic acid fragments; i.e., Fragment 1, Fragment 2, Fragment 3, Fragment 4, Fragment 5, PgapGFP1, and GFP2, had been inserted in that order into the multiple cloning site of pUC-Cen2Paox1MCSTgap1Zeo linearized via MluI treatment in all samples.

On the basis of the results above, the present inventors found that self-circularization of a vector could be completely suppressed by inactivating a gene encoding a DNL4 protein associated with non-homologous end joining. The present inventors also found that vectors could be assembled efficiently in a yeast cell in which the gene is inactivated. Specifically, the present inventors succeeded in efficiently assembling vectors in yeast by a transformation technique comprising a step of introducing two or more types of vectors into a methanol-utilizing yeast strain in which the DNL4 gene associated with non-homologous end joining has been inactivated.

### Production Example 7: Construction of Vector for Genome Integration

A nucleic acid fragment comprising the green fluorescent protein gene (SEQ ID NO: 23) to which the GAP promoter (SEQ ID NO: 4) has been ligated was prepared by PCR using synthetic DNA as a template, primer 45 (SEQ ID NO: 66), and primer 46 (SEQ ID NO: 67). Thus, 1,309-bp Fragment G1 (SEQ ID NO: 68) was constructed.

A nucleic acid fragment comprising a nucleotide sequence of nucleotides 1 to 238 of the CCA38473 terminator downstream of the AOX1 terminator was prepared by PCR using synthetic DNA as a template, primer 47 (SEQ ID NO: 69), and primer 48 (SEQ ID NO: 70). Thus, 584-bp Fragment G2 (SEQ ID NO: 71) was constructed.

A nucleic acid fragment comprising the promoter-regulated G418-resistance gene (SEQ ID NO: 22) and the nucleotide sequence of pUC19 downstream of the nucleotide sequence of nucleotides 239 to 600 of the CCA38473 terminator was prepared by PCR using synthetic DNA as a template, primer 49 (SEQ ID NO: 72), and primer 50 (SEQ ID NO: 73). Thus, 4,095-bp Fragment G3 (SEQ ID NO: 74) was constructed.

Fragment G1 is designed to comprise, at the upper end, a 50-bp nucleotide sequence exhibiting 100% sequence identity to the lower end of Fragment G3 and, at the lower end, a 46-bp nucleotide sequence exhibiting 100% sequence identity to the upper end of Fragment G2.

Fragment G2 is designed to comprise, at the upper end, a 46-bp nucleotide sequence exhibiting 100% sequence identity to the lower end of Fragment G1 and, at the lower end, a 238-bp nucleotide sequence exhibiting 100% sequence identity to the CCA38473 terminator of the genome.

Fragment G3 is designed to comprise, at the upper end, a 362-bp nucleotide sequence exhibiting 100% sequence identity to the CCA38473 terminator of the genome and, at the lower end, a 50-bp nucleotide sequence exhibiting 100% sequence identity to the upper end of Fragment G1.

The Figure shows the correlation of the vectors.

### Comparative Example 4: Generation of Transformed Yeast, Calculation of Transformation Efficiency, and Calculation of Assembly Efficiency

In order to integrate the assembled vectors into the genome, Fragment G1, Fragment G2, and Fragment G3 constructed in Production Example 7 were used to transform the wild-type strain of the Komagataella pastoris strain ATCC76273 in the same manner as in Comparative Example 2.

The competent cell suspension obtained in Comparative Example 2 (60 μl), a solution of Fragment G1 constructed in Production Example 7 (0.3 pmol), a solution of Fragment G2 constructed in Production Example 7 (0.3 pmol), and a solution of Fragment G3 constructed in Production Example 7 (0.015 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and the supernatant was discarded. The yeast culture was resuspended in 1 ml of YNB solution (0.17% Yeast Nitrogen Base without Amino Acids and Ammonium Sulfate (Becton, Dickinson and Company) and 0.1% sodium glutamate (Nacalai Tesque Inc.)), the yeast culture was harvested (3000×g, 5 minutes, 20° C.), and 950 μl of the supernatant was discarded. The yeast culture was resuspended in a remaining solution and spread on a selective SD G418 agar plate (0.17% Yeast Nitrogen Base without Amino Acids and Ammonium Sulfate (Becton, Dickinson and Company), 0.1% sodium glutamate (Nacalai Tesque Inc.), 2% glucose, 1.5% agarose, and 0.05% G418 disulfate salt (Nacalai Tesque Inc.)). A strain that grew in static culture for 3 days at 30° C. was selected, and transformation efficiency was calculated (the number of colonies per gag of vector, cfu/l g) (Table 6, Condition 15).

The 24 transformed yeast strains obtained under Condition 15 were inoculated on a selective SD G418 agar plate and subjected to static culture at 30° C. for 1 day. In order to confirm that vectors had been assembled, chromosome DNA was obtained from the grown strains using Kaneka Easy DNA Extraction Kit Version 2 (Kaneka Corporation) and subjected to PCR with the use thereof as a template, primer 51 (SEQ ID NO: 75) as a forward primer upstream of the CCA38473 terminator, and primer 52 (SEQ ID NO: 76) as a reverse primer downstream of the CCA38473 terminator (Figure). The PCR product was electrophoresed, and assembly efficiency was calculated based on the number of samples in which a band of the size of interest (about 7 kbp) had developed (Table 6, Condition 15).

### Example 5: Generation of Transformed Yeast, Calculation of Transformation Efficiency, and Calculation of Assembly Efficiency

In order to integrate the assembled vectors into the genome. Fragment G1, Fragment G2, and Fragment G3 constructed in Production Example 7 were used to transform the histidine auxotrophic transformed yeast strain Δdn14Δhis4 constructed in Example 1 in which the gene comprising the nucleotide sequence as shown in SEQ ID NO: 25 (DNL4 gene) had been inactivated in the same manner as in Example 3.

The competent cell suspension obtained in Example 3 (60 μl), a solution of Fragment G1 constructed in Production Example 7 (0.3 pmol), a solution of Fragment G2 constructed in Production Example 7 (0.3 pmol), and a solution of Fragment G3 constructed in Production Example 7 (0.015 pmol) were mixed with each other, and the mixture was transferred into an electroporation cuvette (disposable cuvette electrode, electrode gap of 2 mm, BM Equipment Co., Ltd.). After the mixture was subjected to electroporation at 7.5 kV/cm, 25 ρF, and 200Ω, the resulting cells were suspended in 1 ml of YPD medium and allowed to stand for 1 hour at 30° C. After the resultant was allowed to stand for 1 hour, the yeast culture was harvested (3000×g, 5 minutes, 20° C.), and the supernatant was discarded. The yeast culture was resuspended in 1 ml of YNB solution (0.17% Yeast Nitrogen Base without Amino Acids and Ammonium Sulfate (Becton, Dickinson and Company) and 0.1% sodium glutamate (Nacalai Tesque Inc.)), the yeast culture was harvested (3000×g, 5 minutes, 20° C.), and 950 μl of the supernatant was discarded. The yeast culture was resuspended in a remaining solution and spread on a selective SD G418His agar plate (0.17% Yeast Nitrogen Base without Amino Acids and Ammonium Sulfate (Becton, Dickinson and Company), 0.1% sodium glutamate (Nacalai Tesque Inc.), 2% glucose, 1.5% agarose, 0.05% G418 disulfate salt (Nacalai Tesque Inc.), and 0.004% L-histidine (Wako Pure Chemical Corporation)). A strain that grew in static culture for 3 days at 30° C. was selected, and transformation efficiency was calculated (the number of colonies per μg of vector; cfu/μg) (Table 6, Condition 16).

The 24 transformed yeast strains obtained under Condition 16 were inoculated on a selective SD G418His agar plate and subjected to static culture at 30° C. for 1 day. In order to confirm that vectors had been assembled, chromosome DNA was obtained from the grown strains using Kaneka Easy DNA Extraction Kit Version 2 (Kaneka Corporation) and subjected to PCR with the use thereof as a template, primer 51 (SEQ ID NO: 75) as a forward primer upstream of the CCA38473 terminator, and primer 52 (SEQ ID NO: 76) as a reverse primer downstream of the CCA38473 terminator (Figure). The PCR product was electrophoresed, and assembly efficiency was calculated based on the number of samples in which a band of the size of interest (about 7 kbp) had developed (Table 6. Condition 16).

TABLE 6

| | | | | Molar ratio | Transformation | Assembly |
|---|---|---|---|---|---|---|
| | | | | (Vector | efficiency | efficiency |
| Condition | Host | Vector (1) | Vector (2) | (1):Vector (2)) | (cfu/μg) | (%) |
| 15 | Wild-type | Fragment G1, | Fragment G3 | 20:1 | 6.7 × 10³ | 62.5 |
| | | Fragment G2 | | | | |
| 16 | Δdnl4Δhis4 | Fragment G1, | Fragment G3 | 20:1 | 5.9 × 10³ | 100 |
| | | Fragment G2. | | | | |

As a result, the assembly efficiency of Condition 15 attained with the use of Fragment G1, Fragment G2, and Fragment G3 was found to be 62.8%. This indicates that vectors are not efficiently assembled in yeast due to, for example, self-circularization of a vector that had occurred because of non-homologous end joining activity of the wild-type strain. Also, the vector may have been integrated into an unintended site of the genome.

In contrast, the assembly efficiency of Condition 16 attained with the use of Fragment G1, Fragment G2, and Fragment G3 was as high as 100%. This indicates that vectors are assembled with each other efficiently in yeast and the assembled vectors are integrated into an intended site of the genome because the Δdn14Δhis4 strain has lost non-homologous end joining activity upon inactivation of the gene comprising the nucleotide sequence as shown in SEQ ID NO: 25 (DNL4 gene) and self-circularization of a vector has been completely suppressed.

The results demonstrated above indicate that the present inventors succeeded in efficiently assembling vectors in yeast by inactivating the gene encoding the DNL4 protein associated with non-homologous end joining by a transformation technique aimed at genome integration.

All publications, patents, and patent applications cited herein are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the present invention should be limited only by the attached claims.

What is claimed is:

1. A method for assembling two or more types of vectors comprising introducing the two or more types of vectors into a methanol-utilizing yeast strain,
wherein the methanol-utilizing yeast strain comprises a DNL4 gene that is inactivated, and
wherein the two or more types of vectors are assembled in a transformant of the methanol-utilizing yeast strain.

2. The method according to claim 1, wherein the DNL4 gene is selected from the group consisting of:
a gene comprising the nucleotide sequence of SEQ ID NO: 25;
a gene comprising a nucleotide sequence of a nucleic acid hybridizing under stringent conditions to a nucleic acid comprising a nucleotide sequence complementary to the nucleotide sequence of SEQ ID NO: 25;
a gene comprising a nucleotide sequence having 85% or higher sequence identity to the nucleotide sequence of SEQ ID NO: 25; and
a gene encoding an amino acid sequence having 85% or higher sequence identity to the amino acid sequence of SEQ ID NO: 24.

3. The method according to claim 1, wherein the methanol-utilizing yeast strain is a yeast strain belonging to the genus Komagataella or a yeast strain belonging to the genus Ogataea.

4. The method according to claim 1, wherein the two or more types of vectors each comprise a nucleotide sequence having 85% or higher sequence identity to one another.

5. The method according to claim 1, wherein at least one of the two or more types of vectors comprises an autonomously replicating sequence (ARS).

6. The method according to claim 1, wherein at least one of the two or more types of vectors is an autonomously replicating vector.

7. The method according to claim 6, wherein the autonomously replicating vector further comprises an autonomously replicating sequence (ARS) and/or a centromeric DNA sequence derived from a yeast strain belonging to the genus Komagataella or a yeast strain belonging to the genus Ogataea.

8. The method according to claim 1, further comprising isolating the assembled vectors from the transformant.

9. The assembled vectors obtained by the method according to claim 8.

10. The method according to claim 8, further comprising producing a transformant by performing transformation with the isolated assembled vectors.

11. The transformant transformed with the isolated assembled vectors by the method according to claim 10.
